(19) United States
(12) Patent Application Publication
(10) Pub. No.: US 2010/0281567 A1
(43) Pub. Date: Nov. 4, 2010

(54) TRANSGENIC PLANTS HAVING ALTERED LEVELS OF AROMATIC AMINO ACIDS AND METABOLITES DERIVED THEREFROM

(76) Inventors: Gad Galili, Rehovot (IL); Vered Tzin, Rehovot (IL); Asaph Aharoni, Rehovot (IL); Sergey Malitsky, Rehovot (IL)

Correspondence Address: FENNEMORE CRAIG, 3003 NORTH CENTRAL AVENUE, SUITE 2600, PHOENIX, AZ, 85012, US

(21) Appl. No.: 12/745,853

(22) PCT Filed: Dec. 3, 2008
(86) PCT No.: PCT/IL08/01569

§ 371 Date: Jul. 8, 2010

Related U.S. Application Data
(60) Provisional application No. 60/992,106, filed on Dec. 4, 2007.
(60) Provisional application No. 61/095,638, filed on Sep. 10, 2008.

(51) Int. Cl. A01H 1/00; C12N 5/10; A01H 5/00

(52) U.S. Cl. 800/278; 435/419; 800/298

(57) ABSTRACT

The present invention relates to means and methods for altering the level of at least one of the aromatic amino acids phenylalanine, tryptophan and tyrosine and secondary metabolites in plants. Particularly, the present invention discloses transgenic plants comprising polynucleotides encoding chorismate mutase and prephenate dehydratase enzymes, having elevated amounts of at least one of phenylalanine, tyrosine and modified amount of at least one secondary metabolite derived therefrom, and reduced amount of tryptophan and at least one secondary metabolite derived from tryptophan.

### FIELD OF THE INVENTION

The present invention relates to means and methods for altering the level of aromatic amino acids and secondary metabolites derived therefrom in plants, particularly to transgenic plants comprising polynucleotides encoding chorismate mutase and/or prephenate dehydratase enzymes.

### BACKGROUND OF THE INVENTION

The essential aromatic amino acids phenylalanine, tryptophan and tyrosine are synthesized in plants from chorismate (FIG. 1), which is thus the precursor for a variety of secondary metabolites produced from these amino acids (Verberne et al., 2007 J Biotechnol 128:72-79). The biochemical pathway leading to phenylalanine and tyrosine synthesis from chorismate in plants is still largely unknown. In fact, the only known biochemical step in this pathway is the conversion of chorismate to prephenate catalyzed by the enzyme Chorismate Mutase (CM). Several genes encoding CM isozymes have been cloned from the model plant Arabidopsis (Mobley et al., 1999 Gene 240:115-123). Notably, the isozymic polypeptides encoded by these genes either contain or lack putative plastid transit peptide, indicating that CM activities may reside both in the cytosol and in the plastid of plant cells (Eberhard et al., 1996 Plant J 10:815-821; Herrmann and Weaver, 1999 Physiol Plant Mol Biol 50:473-503). The first step in the biosynthesis of tryptophan from chorismate is the conversion of chorismate into anthranilate, catalyzed by the enzyme Anthranilate Synthase (AS) (FIG. 1).

In nature, microorganisms use at least two different metabolic routes for the synthesis of phenylalanine from prephenate, utilizing either phenylpyruvate (PPY) or arogenate (also known as pretyrosine) as intermediates. Phenylalanine biosynthesis through the PPY pathway includes the conversion of prephenate into PPY by a prephenate dehydratase (PDT) enzyme and subsequent conversion of PPY into phenylalanine by an aromatic amino acid aminotransferase enzyme. This pathway is found, for example, in Escherichia coli (E. coli) and Bacillus subtilis. Phenylalanine biosynthesis through the arogenate pathway includes the conversion of prephenate into arogenate by a prephenate aminotransferase (PAT) enzyme and the subsequent conversion of arogenate to phenylalanine by an arogenate dehydratase (ADT) enzyme. This pathway is found for instance in some cynobacteria, coryneform bacteria and spore forming actinomycetes. Some organisms, such as Pseudomonas aeruginosa and Erwinia herbicola, possess a specific cyclohexadienyl dehydratase enzyme, which was reported to use either PPY or arogenate as substrates. Arogenate may be also converted to tyrosine by arogenate dehydrogenase.

Although plants, like microorganisms, use CM to convert chorismate into prephenate (Eberhard et al., 1996, ibid; Mobley et al., 1999 Gene 240:115-123), the subsequent enzymatic steps for the synthesis of phenylalanine from prephenate are still poorly understood in plants. Several lines of indirect evidence have suggested that plants can synthesize both phenylalanine and tyrosine from arogenate. A PAT enzymatic activity, converting prephenate into arogenate, has been reported in plants. Yet, no plant gene encoding a polypeptide having such an activity has so far been reported to be cloned from any plant species (Ehlting et al., 2005 Plant J 42:618-640). The conversion of arogenate into phenylalanine by ADT has also been demonstrated in tobacco chloroplasts, spinach chloroplasts and etiolated sorghum seedlings. Also in this case, no gene encoding ADT has so far been reported to be cloned from any plant species (Ehlting et al., 2005, ibid).

In plant cells, arogenate was detected as an intermediate metabolite as well as an activity of arogenate dehydrogenase (ADS), which converts arogenate to tyrosine. This activity has been demonstrated in tobacco, maize, sorghum and the photosynthetic microorganism Chlorella sorokiniana. Moreover, two genes encoding ADS have been described in the model plant Arabidopsis (At1g15710 and At5g34930) (Rippert and Matringe, 2002 Eur J Biochem 269:4753-4761). These evidence provide further support for the utilization of agrogenate in phenylalanine synthesis in plant cells.

It has been suggested recently that the Arabidopsis genome possesses several genes with limited homology to the PDT genes of microorganisms (PDT1-At2g27820, At1g08250, At1g11790, At3g07630, At3g44720 and At5g22630), which synthesize PPY. This report also showed that the PDT1 protein is located in the cytosol, and is produced in etiolated seedlings, with a specific role in blue light-mediated synthesis of PPY (Warpeha et al., 2006 Plant Physiol 140:844-855). Yet, as opposed to experimental evidence supporting the use of arogenate in phenylalanine biosynthesis, there is no experimental evidence showing the potential function of PPY as an intermediate of phenylalanine biosynthesis in plants. Nevertheless, in several plant species, PPY has been shown to be a precursor for a number of secondary metabolites such as the scent metabolite phenylacetaldehyde, indicating that PPY is produced in at least in some plant species (Watanabe et al., 2002 Biosci Biotechnol Biochem 66:943-947; Kaminaga et al., 2006 J Biol Chem 281:23357-23366).

The biosynthesis of phenylalanine from chorismate in E. coli is catalyzed by a single bifunctional polypeptide designated P-protein that is encoded by a single gene, which contains both CM and PDT activities (Romero et al., 1995 Phytochemistry 40:1015-1025). CM catalytic activity is located at amino acids 1-109, while PDT activity is located at amino acids 101-285 (Zhang et al., 1998 J Biol Chem 273:6248-6253). The E. coli P-protein also contains an additional C-terminal domain, called the R-domain (amino acids 286-386), which is responsible for the sensitivity of the bifunctional CM/PDT enzyme to feedback inhibition by phenylalanine (Zhang et al., 1998, ibid). Truncated CM/PDT protein containing only amino acids 1-285 or amino acids 1-300 retain CM and PDT activities, but exhibit no feedback inhibition by Phenylalanine. Expression of such truncated CM/PDT polypeptides in E. coli also led to phenylalanine overproduction.

Plants produce a large array of metabolites, called secondary metabolites. Many plant secondary metabolites have a significant commercial value associated with their multiple benefits to humans, such as improving human health and enhancing the natural colors, tastes and aromas of human foods. On the other hand, several secondary metabolites are known to be nutritionally harmful to human.

Siebert et al. (1996 Plant Physiol 112:811-819) have described transgenic plant expressing the ubiC gene of E. coli, a gene encoding chorismate pyruvatelyase that converts chorismate into 4-hydroxybenzoate (4HB), but does not lead to the production of the aromatic amino acids phenylalanine, tyrosine and tryptophan. The transgenic plants accumulated high levels of 4HB as beta glucosides. 4HB is a precursor to shikonin, a naphthoquinone pigment with antibacterial, antiphylogistic and wound healing properties.

U.S. Pat. No. 6,303,847 discloses techniques for controlling the expression of genes relating to the biosynthesis of phenylpropanoid, particularly an isolated and purified DNA encoding a transcription factor controlling a phenylpropanoid biosynthesis pathway and transgenic plants comprising same.

U.S. Pat. No. 7,154,023 discloses transgenic plants with altered levels of phenolic compounds, produced by altering the levels of one or more phenolic compounds that are intermediates or final products of the plant phenylpropanoid pathway. Expression constructs comprising nucleic acid encoding a transactivator protein comprising the myb domain of the maize “ZmMyb-IF35” or a transgene which encodes an antisense ZmMyb-IF35 RNA are provided.

U.S. Pat. No. 7,189,895 discloses methods of increasing isoflavonoid production in isoflavonoid-producing plants by transforming plants with at least one construct expressing at least a portion of a flavanone 3-hydroxylase, a C1 myb transcription factor, and an R-type myc transcription factor that regulate expression of genes in the phenylpropanoid pathway.

U.S. Pat. No. 7,332,642 discloses process for the production of fine chemicals, particularly vitamin E, vitamin K and/or ubiquinone, by genetically modifying the shikimate pathway in organisms, particularly in plants. That patent discloses the use of chorismate mutase, prephenate dehydrogenase or a combination thereof for modifying the shikimate pathway towards the production of 4-hydroxyphenylpyruvate, leading to the desired increase in vitamin E, vitamin K and/or ubiquinone content.

U.S. Patent Application Publication No. 20060236421 discloses methods for modulating the rate of production and accumulation of secondary metabolites, e.g., alkaloid, terpenoid or phenylpropanoid compounds. Also disclosed are compositions useful in such methods, e.g., a plant containing a recombinant nucleic acid that is effective for reducing the level of general DNA methylation.

However, nowhere in the background art is it disclosed or suggested to utilize the alteration of the essential aromatic amino acids levels for controlling the synthesis of secondary metabolites. Thus, there is a recognized need for, and it would be highly advantageous to have means and methods for controlling the production of phenylalanine, tryptophan and tyrosine that would result in overproduction of highly desired secondary metabolites while reducing the production of harmful metabolites.

### SUMMARY OF THE INVENTION

The present invention relates to means and methods for altering the level of the aromatic amino acids tyrosine, tryptophan and phenylalanine in plants. Particularly, the invention relates to overproduction of the amino acid phenylalanine in transgenic plants, leading to the overproduction of several highly desired plant secondary metabolites, particularly catabolic products of phenylalanine and also of tyrosine, and decreased production of several undesired plant secondary metabolites, particularly glucosinolates (GSs), which are catabolic products of tryptophan.

The present invention is based in part on the unexpected discovery that expression in a plant cell of a modified bacterial bifunctional polypeptide, having chorismate mutase (CM) and prephenate dehydratase (PDT) activities and reduced sensitivity to feedback inhibition by phenylalanine results in overexpression of phenylalanine in the cell, and, moreover, in overexpression of secondary metabolites of the phenylpropanoids group and catabolic products of tyrosine, while synthesis of tryptophan is reduced.

Without wishing to be bound by any particular theory or mechanism of action the highly desired overexpression of phenylalanine and tyrosine catabolic products may be attributed to an increase in the available amount of phenylalanine, which is known to be an essential precursor in the phenylpropanoids and tyrosine biosynthetic pathway.

Thus, according to one aspect, the present invention provides a transgenic plant comprising at least one plant cell comprising an exogenous polynucleotide encoding chorismate mutase (CM) and an exogenous polynucleotide encoding prephenate dehydratase, wherein the transgenic plant has an altered content of at least one aromatic amino acid compared to a corresponding non transgenic plant.

According to certain embodiments, the aromatic amino acid is selected from the group consisting of phenylalanine, tryptophan and tyrosine. According to one embodiment, the transgenic plant contains elevated amount of at least one of phenylalanine and tyrosine compared to the corresponding non transgenic plant. According to another embodiment, the transgenic plant contains reduced amount of tryptophan compared to the corresponding non transgenic plant.

According to certain currently preferred embodiments, the transgenic plant contains elevated amounts of phenylalanine compared to the corresponding non transgenic plant. According to typical embodiments, the transgenic plant contains elevated amounts of phenylalanine and reduced amount of tryptophan compared to the corresponding non transgenic plant.

According to other embodiments, the transgenic plant has an increased amount of at least one catabolic product of phenylalanine compared to the corresponding non transgenic plant. According to one embodiment, the catabolic product of phenylalanine is selected from the group consisting of phenethyl isothiocyanate, 2-phenylethyl glucosinolate, benzyl glucosinolate, phenylacetonitrile, sinapyl alcohol, coniferon, caffeol glucose, acetovanillone, vanillic acid glucoside, coumarate hexose, ferulate hexose or combinations thereof.

Unexpectedly, the present invention now shows that increased levels of phenylalanine in the transgenic plant cell results in an increased amount of catabolic products of tyrosine. Thus, according to certain embodiments, the transgenic plant has an increased amount of at least one catabolic product of tyrosine. According to one embodiment, the catabolic product of tyrosine is selected from the group consisting of homogentistic acid, gamma-tocopherol, gamma-tocotrienol and combinations thereof.

The present invention further shows that overexpression of phenylalanine, while leading to an increase in certain metabolites also results in a decrease in the amount of other metabolites. Without wishing to be bound by any theory or mechanism of action, this pattern of expression may be due to a shift in the pathway towards certain metabolites, leading to reduction in the expression of others. The present invention now discloses that the overexpressed metabolites are highly desired while the down regulated metabolites are undesired plant secondary metabolites.

Thus, according to other embodiments, the transgenic plant has a reduced amount of at least one catabolic product of phenylalanine compared to the corresponding non transgenic plant. According to one embodiment, the catabolic product of phenylalanine is phenylalanine 3-carboxy-2-hydroxy.

According to yet additional embodiments, the transgenic plant has a decreased amount of at least one catabolic product of tryptophan compared to the corresponding non transgenic plant. According to one embodiment, the catabolic product of tryptophan is selected from the group consisting of 1-methoxy-3-indolylmethyl, 6-hydroxyindole-3-carboxylic acid 6-O-beta-D-glucopyranoside, 6-hydroxyindole-3-carboxylic acid beta-D-glucopyranosyl ester, 4-O-(indole-3-acetyl)-D-glucopyranose, tryptophan N-formyl-methyl ester and combinations thereof.

According to certain embodiments, the CM activity, the PDT activity or both enzymatic activities have reduced sensitivity to feedback inhibition by phenylalanine.

According to certain embodiments, the chorismate mutase and the prephenate dehydratase are encoded by a single polynucleotide.

According to other embodiments, the polynucleotides encoding the chorismate mutase, the prephenate dehydratase or a combination thereof are of a prokaryotic origin, typically of a bacterial origin. According to one embodiment, the polynucleotides are of E. coli. According to certain currently preferred embodiments, the polynucleotide encodes C-terminal truncated CM/PDT of E. coli having CM and PDT activities (NCBI Accession No. AAN81570, SEQ ID NO:1).

According to certain embodiments, the polynucleotide comprises nucleic acid sequence corresponding to nucleic acids 1-900 (SEQ ID NO:10) of E. coli PheA gene (NCBI Accession No. AE014075, SEQ ID NO:2) encoding amino acids 1-300 of E. coli CM/PDT protein.

The present invention discloses for the first time that a significant portion of the synthesis of phenylalanine products in a plant cell occurs within the cell plastids. Thus, according to certain embodiments, the polynucleotides encoding chorismate mutase and/or prephenate dehydratase further comprise a nucleic acid sequence encoding a plastid transit peptide. Typically, CM and PDT are encoded by a single polynucleotide and the polynucleotide is so designed that the plastid transit peptide is fused at the carboxy terminus of the encoded polypeptide.

According to particular embodiments, the polynucleotide comprises a nucleic acid sequence as set forth in SEQ ID NO:3. According to other particular embodiments, the encoded polypeptide, containing the pea rbcS3 plastid transit peptide and residues 1-300 of the E. coli CM/PDT protein, has an amino acid sequence as set forth in SEQ ID NO:4.

According to yet other embodiments, the polynucleotides of the present invention are incorporated in a DNA construct enabling their expression in the plant cell. According to one embodiment, the DNA construct comprises at least one expression regulating element selected from the group consisting of a promoter, an enhancer, an origin of replication, a transcription termination sequence, a polyadenylation signal and the like.

According to some embodiments, the DNA construct comprises a promoter. The promoter can be constitutive, induced or tissue specific promoter as is known in the art. According to typical embodiments, the promoter is a constitutive promoter operable in a plant cell. According to another embodiment, the DNA construct further comprises transcription termination and polyadenylation sequence signals.

Optionally, the DNA construct further comprises a nucleic acid sequence encoding a detection marker enabling a convenient detection of the recombinant polypeptides expressed by the plant cell. According to certain embodiments, the DNA construct further comprises a nucleic acid sequence encoding a hemagglutinin (HA) epitope tag. This epitope allows the detection of the recombinant polypeptide by using antibodies raised against the HA epitope tag. According to one embodiment, the DNA construct comprises a nucleic acid sequence as set forth in SEQ ID NO:5, encoding a polypeptide containing the pea rbcS3 plastid transit peptide, residues 1-300 of the E. coli PheA protein and three repeats of the HA epitope tag, having SEQ ID NO:6.

The polynucleotides of the present invention and/or the DNA constructs comprising same can be incorporated into a plant transformation vector.

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the CM and PDT activity of the polypeptide in the context of the present invention of altering the level of at least one aromatic amino acid selected from the group consisting of phenylalanine, tryptophan or tyrosine in a plant cell. Specifically, any active fragments of the active polypeptide or protein as well as extensions, conjugates and mixtures are disclosed according to the principles of the present invention.

The present invention also encompasses seeds of the transgenic plant, wherein plants grown from said seeds comprise at least one cell having an altered content of at least one aromatic amino acids compared to plants grown from seeds of corresponding non transgenic plant. The present invention further encompasses fruit, leaves or any part of the transgenic plant, as well as tissue cultures derived thereof and plants regenerated therefrom.

According to yet another aspect, the present invention provides a method of altering the synthesis of at least one aromatic amino acid in a plant, comprising (a) transforming a plant cell with an exogenous polynucleotide encoding chorismate mutase and an exogenous polynucleotide encoding prephenate dehydratase; and (b) regenerating the transformed cell into a transgenic plant comprising at least one cell having an altered content of at least one aromatic acid compared to a corresponding cell of a non transgenic plant.

The exogenous polynucleotide(s) encoding chorismate mutase and prephenate dehydratase according to the teachings of the present invention can be introduced into a DNA construct to include the entire elements necessary for transcription and translation as described above, such that the polypeptides are expressed within the plant cell.

Transformation of plants with a polynucleotide or a DNA construct may be performed by various means, as is known to one skilled in the art. Common methods are exemplified by, but are not restricted to, Agrobacterium-mediated transformation, microprojectile bombardment, pollen mediated transfer, plant RNA virus mediated transformation, liposome mediated transformation, direct gene transfer (e.g. by microinjection) and electroporation of compact embryogenic calli. According to one embodiment, the transgenic plants of the present invention are produced using Agrobacterium mediated transformation.

Transgenic plants comprising the polynucleotides of the present invention may be selected employing standard methods of molecular genetics, as are known to a person of ordinary skill in the art. According to certain embodiments, the transgenic plants are selected according to their resistance to an antibiotic. According to one embodiment, the antibiotic serving as a selectable marker is one of the group consisting of cefotaxime, vancomycin and kanamycin.

According to other aspects the present invention relates to the transgenic plants generated by the methods of the present invention as well as to their seeds, fruits, roots and other organs or isolated parts thereof.

Other objects, features and advantages of the present invention will become clear from the following description and drawings.

### BRIEF DESCRIPTION OF THE FIGURES

FIG. 1 shows schematic diagram of the biosynthesis of aromatic amino acids in plants. The schemes describe the synthesis of phenylalanine, tyrosine and tryptophan from chorismate. Dashed grey arrows with a minus sign represent feedback inhibition loops of chorismate mutase by the amino acids tyrosine and phenylalanine. Dashed black arrows represent secondary metabolite pathways.

FIG. 2 is a schematic diagrams of the chimeric genes used in the present invention. FIG. 2A: 35S:PRO-PheA*-HA and FIG. 2B: 35S:PRO-TP-PheA*-HA. PheA*-truncated CM/PDT polypeptide, that is phenylalanine feedback-insensitive (Zhang et al., 1998); TP—transit peptide; 3HA-three repeats of hemagglutinin epitope tag.

FIG. 3 shows an immunoblot analysis of PheA* gene product in transgenic Arabidopsis plants, using Western blot analysis with anti HA antibodies. FIG. 3A: Plants transformed with 35S:PRO-PheA*-3HA. FIG. 3B: Plants transformed with 35S:PRO-TP-PheA*-3HA. The different numbers on top of the panels represent the independently transformed lines. TP-PheA*-HA: precursor polypeptide containing the PheA* polypeptide and a transit peptide; PheA*-HA, mature polypeptide.

FIG. 4 illustrates fold increase of phenylalanine extracted form leaves of different 10 days old transgenic plants expressing the 35S:PRO-PheA*-3HA and 35S:PRO-TP-PheA*-3HA DNA constructs. Metabolites levels (fold increase above levels in control plants transformed with empty vector) were calculated from three to six independent replicates. p5, p9 and p17 represent plant lines independently transformed with the 35S:PRO-TP-PheA*-3HA DNA construct. c11, c16 and c20 represent plant lines independently transformed with the DNA construct 35S:PRO-PheA*-3HA. Asterisks on top of the histograms represent significant difference (p<0.05) from the control plants (con).

FIG. 5 illustrates fold increase of catabolic products of phenylalanine and tyrosine in different transgenic plants expressing the 35S:PRO-PheA*-3HA and 35S:PRO-TP-PheA*-3HA DNA constructs. Metabolites levels (fold increase above level in control plants transformed with empty vector) were calculated from five to six independent replicates. FIG. 5A: homogentistic acid. FIG. 5B: phenethyl isothiocyanate. These metabolites were identified by commercially available standards (Sigma). p5, p9 and p17 represent plant lines independently transformed with the DNA construct 35S:PRO-TP-PheA*-3HA. c11, c16 and c20 represent plant lines independently transformed lines with the DNA construct 35S:PRO-PheA*-3HA. Asterisks on top of the histograms represent significant difference (p<0.05) from the control plants (con).

FIG. 6 shows alteration in the level of selected metabolites in transgenic plants expressing the 35S:PRO-TP-PheA*-3HA compared to control plants. Metabolites were detected either by LC-MS, GC-MS or HPLC. The level of each metabolite is an average of five independent replicates. Polar secondary metabolites were detected by LC-MS apparatus (FIG. 6a-f, h and k-s). Tocopherol and tocotrienol (FIG. 6t and u) were detected by HPLC and were measured using standard curves (marked **). 2-Phenethyl isothiocyanate (FIG. 6g), Phenylacetonitrile (FIG. 6i) and Homogenitisic acid (FIG. 6j) were detected by GC-MS apparatus (marked *). The selected metabolite level was significantly different in all three transgenic genotype (p<0.05) compared to the control plants.

FIG. 7 is a principal component analysis (PCA) of metabolite of different transgenic plants expressing the 35S:PRO-PheA*-3HA or 35S:PRO-TP-PheA*-3HA DNA constructs. The analysis was based on three to six replicates for the different genotypes. Each metabolite was normalized by dividing the response level by the median, and this value was transformed to log₁₀. The analysis of the data was performed using TMEV4 software (Saeed et al. 2003 Biotechniques 34:374-378; Scholz et al. 2004 Bioinformatics 20:2447-2454). The PCA is represented by component 1 and 2, analyzed from the response level of 90 metabolites, integrated by Xcalibur software v.1.4 (ThermoFinnigan, Manchester, UK).

FIG. 8 illustrates fold increase of phenylalanine in different transgenic plants expressing the 35S:PRO-TP-PheA*-3HA genes. Cauline and rosette leaves of two month old Arabidopsis plants were collected and extracted for GC-MS analysis. Metabolites levels (fold increase above levels in control plants transformed with empty vector) were calculated from three independent replicates. p3, p5, p9, p16, p17, p20, p24, p28-independently transformed lines with the 35S:PRO-TP-PheA*-3HA construct. Asterisks on top of the histograms represent significant difference (p<0.05) from the control plants (con).

FIG. 9 shows the effect of the HPPD inhibitor Isoxaflutole on growth of control and transgenic Arabidopsis plants expressing the 35S:PRO-TP-PheA*-3HA construct. Plants were grown on MS medium in the presence of 10 ppb Isoxaflutole. Control plants, transformed with empty pART27 vector (con) developed chlorotic symptoms, not seen in the transgenic plants expressing the 35S:PRO-TP-PheA*-3HA construct. FIG. 9A: Chlorophyll content. FIG. 9B: Photograph of the plants.

FIG. 10 illustrates the effect of tryptophan analog on transgenic plants expressing: (i) the 35S:PRO-TP-PheA*-3HA gene (p5, p9; p17); (ii) dominant mutation, resistant to tryptophan analogs due to high levels of tryptophan (5× normal) and anthranilate synthase (2× normal), amt1-1; (iii) control plants expressing an empty Ti vector (con); and (iv) wild type Arabidopsis Colombia ecotype (col). FIG. 10A: Photograph of plants growing for three weeks on zero and 100 μM 5-methyl tryptophan (5MT). FIG. 10B: Quantitative analysis of the number of seedlings whose growth was arrested by 5MT.

FIG. 11 shows phenotypes of the PheA* plants and susceptibility to the medium phenylalanine content. FIG. 11A: Morphological phenotype of the control genotype (a, e) and the PheA* expressing genotypes p5 (b, f), p9 (c, g) and p17 (d, h). Plants were at the age of 10 days (a-d) and two months (e-f). FIG. 11B: Seeds of the above control and transgenic plants were germinated on media lacking (left) or containing (right) 4 mM Phe.

### DETAILED DESCRIPTION OF THE INVENTION

The present invention relates in general to the growing interest in naturally derived metabolites for the pharmaceutics, cosmetics and food industries, as well as for improved agricultural products.

The present invention discloses transgenic plants transformed with exogenous nucleic acids encoding chorismate mutase (CM) and prephenate dehydratase (PDT). In E. coli, these enzyme activities are shown by a bi-functional protein, encoded by a single gene, PheA. A truncated C-terminal protein, designated PheA* has been shown to be insensitive to negative feedback by phenylalanine (Zhang et al. 1998, ibid).

The present invention now shows that transgenic plants expressing the PheA* gene produce increased amount of phenylalanine compared to corresponding non transgenic plants. Moreover, the present invention now shows that the production of a bacterial PheA* polypeptide in transgenic plants, particularly within the plastid of the plant cell leads to over production of secondary metabolites, which require phenylalanine and/or tyrosine and/or intermediate compounds produced through the prephenate-phenylpyruvate-phenylalaine pathway for their biosynthesis. Particularly, the second metabolites include phenylpropanoids, for example phenethyl isothiocyanate and tyrosine catabolic metabolites, for example homogentistic acid. The present invention also shows that the biosynthesis of tryptophan in these transgenic plants is reduced compared to control plants expressing an empty Ti vector with no PheA* construct.

The present invention also provides a method of producing a transgenic plant having altered content of at least one of the aromatic amino acids phenylalanine, tryptophan and tyrosine as compared to a corresponding non transgenic plant. Also provided by the present invention are plant cells, comprising exogenous nucleic acids encoding chorismate mutase and prephenate dehydratase and plant seeds and progenies obtained from the transgenic plants.

The present invention makes a significant contribution to the art by providing new strategies to engineer plants having the capability to modify the production of secondary metabolites. The present invention utilizes the prephenate-phenylpyruvate pathway, not previously shown to be active in plant, for over production of phenylalanine. This pathway is clearly distinct from the shikimate pathway, the modification of which was previously disclosed for altering the level of secondary metabolites in plants

The plants of the present invention are capable of overproducing secondary metabolites required for their beneficial characterizations, which are naturally produced by the plant in insufficient amounts to be used commercially, while having decreased levels of harmful secondary metabolites.

### DEFINITIONS

The term “plant” is used herein in its broadest sense. It includes, but is not limited to, any species of woody, herbaceous, perennial or annual plant. It also refers to a plurality of plant cells that are largely differentiated into a structure that is present at any stage of a plant's development. Such structures include, but are not limited to, a root, stem, shoot, leaf, flower, petal, fruit, etc.

The term “phenylalanine catabolic product(s)” refer to classes of plant-derived organic compounds that are biosynthesized from the amino acid phenylalanine, particularly phenylpropanoids. The phenylpropanoids have a wide variety of functions in the plant, including defense against herbivores, microbial attack, or other sources of injury; as structural components of cell walls; as protection from ultraviolet light; as pigments; and as signaling molecules.

The term “tyrosine catabolic product(s)” refer to classes of plant-derived organic compounds known to be synthesized from the amino acid tyrosine, for example homogentisic acid and gamma-tocopherol.

As used herein the term “gulcosinolates” refer to β-thioglucoside-N-hydroxysulfates, which are nitrogen- and sulfur-containing plant specialized metabolites.

The term “chorismate mutase” as used herein refers to a protein having the enzymatic activity of converting chorismate mutase to prephenate.

The term “prephenate dehydrates” as used herein refers to a protein having the enzymatic activity of converting prephenate to phenylpyruvate.

The terms “E. coli P-protein”, “E. coli CM/PDT protein/polypeptide” and “E. coli PheA” are used herein interchangeably, referring to a bifunctional polypeptide having the amino acid sequence set forth in SEQ ID NO:1, which contains both CM and PDT activities. The protein is encoded by a single gene having the nucleic acid sequence set forth in SEQ ID NO:2.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

An “isolated” nucleic acid molecule is one that is substantially separated from other nucleic acid molecules which are present in the natural source of the nucleic acid (i.e., sequences encoding other polypeptides). Preferably, an “isolated” nucleic acid is free of some of the sequences which naturally flank the nucleic acid (i.e., sequences located at the 5′ and 3′ ends of the nucleic acid) in its naturally occurring replicon. For example, a cloned nucleic acid is considered isolated. A nucleic acid is also considered isolated if it has been altered by human intervention, or placed in a locus or location that is not its natural site, or if it is introduced into a cell by agroinfection. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

The term “recombinant” refers to an artificial combination of two otherwise separated segments of sequence, e.g., by chemical synthesis or by the manipulation of isolated segments of nucleic acids by genetic engineering techniques.

The term “construct” as used herein refers to an artificially assembled or isolated nucleic acid molecule which includes the gene of interest. In general a construct may include the gene or genes of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. New promoters of various types useful in plant cells are constantly being discovered; numerous examples may be found in Okamuro J K and Goldberg R B (1989) Biochemistry of Plants 15:1-82.

As used herein, the term an “enhancer” refers to a DNA sequence which can stimulate promoter activity, and may be an innate element of the promoter or a heterologous element inserted to enhance the level or tissue-specificity of a promoter.

The term “expression”, as used herein, refers to the production of a functional end-product e.g., an mRNA or a protein.

The term “transgenic” when used in reference to a plant or seed (i.e., a “transgenic plant” or a “transgenic seed”) refers to a plant or seed that contains at least one heterologous transcribeable gene in one or more of its cells. The term “transgenic plant material” refers broadly to a plant, a plant structure, a plant tissue, a plant seed or a plant cell that contains at least one heterologous gene in at least one of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of the protein (e.g. β-glucuronidase) encoded by the exogenous polynucleotide.

The term “transient transformant” refers to a cell which has transiently incorporated one or more exogenous polynucleotides. In contrast, the term “stable transformation” or “stably transformed” refers to the introduction and integration of one or more exogenous polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA. It is to be understood that a plant or a plant cell transformed with the nucleic acids, constructs and/or vectors of the present invention can be transiently as well as stably transformed. The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

### PREFERRED MODES FOR CARRYING OUT THE INVENTION

According to one aspect, the present invention provides a transgenic plant comprising at least one plant cell comprising an exogenous polynucleotide encoding chorismate mutase (CM) and an exogenous polynucleotide encoding prephenate dehydratase (PDT), wherein the transgenic plant has an altered content of at least one aromatic amino acid compared to a corresponding non transgenic plant. According to certain embodiments, the aromatic amino acid is selected from the group consisting of phenylalanine, tryptophan and tyrosine. According to one embodiment, the transgenic plant contains elevated amount of at least one of phenylalanine and tyrosine compared to the corresponding non transgenic plant. According to another embodiment, the transgenic plant contains reduced amount of tryptophan compared to the corresponding non transgenic plant. According to certain embodiments, the transgenic plant further overexpresses several secondary metabolites derived from phenylalanine and/or phenylpyruvate and/or tyrosine. According to other embodiment, the transgenic plants produce reduced amounts of undesired plant secondary metabolites, particularly GSs, which are catabolic products of tryptophan. According to typically embodiments, the transgenic plant contains elevated amounts of phenylalanine and reduced amounts of tryptophan.

Various DNA constructs may be used to obtain altered expression of at least one of the aromatic amino acids phenylalanine, tryptophan and tyrosine in a plant cell according to the teachings of the present invention.

According to certain embodiments, each of the polynucleotides encoding chorismate mutase and prephenate dehydratase is included in a separate DNA construct comprising the necessary elements for the polynucleotide expression.

According to other embodiments, the DNA construct includes a single transcribable polynucleotide sequence encoding for a polypeptide having chorismate mutase and prephenate dehydratase activities. According to certain currently preferred embodiments, the encoded polypeptide is insensitive to the negative feedback imposed by elevated amounts of phenylalanine in the cell. According to current embodiments, the present invention utilizes the coding DNA sequence of the E. coli feedback-insensitive PheA (designated PheA*). This sequence (SEQ ID NO:10) comprises nucleic acids 1-900 of the E. coli PheA gene (SEQ ID NO:2), which encodes amino acids 1-300 (SEQ ID NO:4) of the E. coli PheA enzyme, having 386 amino acids (NCBI accession number AAN81570; SEQ ID NO:1).

In E. coli, the 300 amino acid segment (PheA*) retains full mutase and dehydratase activity, but exhibits no feedback inhibition (Zhang et al., 1998, ibid).

Many classes of molecules such as fatty acids and terpenes that are required in the normal development and function of the plant cell are synthesized within the cell plastids. To examine whether the synthesis of phenylalanine and phenylpropanoids takes place in the cytosol or within a plastid, two DNA constructs were designed: one construct comprising a transcribable polynucleotide encoding PheA*, and another construct further comprising an operably linked polynucleotide encoding plastid transit peptide. Any plastid transit peptide as is known in the art may be used according to the teaching of the present invention. According to certain embodiments, the transit peptide is a pea rbsS3 plastid transit peptide encoded by a polynucleotide having SEQ ID NO:9. As exemplified hereinbelow, phenylalanine level in plants expressing the plastidic PheA* showed significantly higher levels of phenylalanine than plants expressing PheA* in the cytosol. Furthermore, in plant cells expressing PheA* in the plastids, not only the levels of phenylalanine were significantly higher, but also elevated levels of the phenylalanine and tyrosine catabolic products were detected.

Lignins are aromatic polymers that are present mainly in secondarily thickened plant cell walls and are one of the most abundant organic compounds in the terrestrial biosphere. An increasing number of examples illustrate that lignin engineering can improve the processing efficiency of plant biomass for pulping, forage digestibility and biofuels (Vanholme et al 2008 Current Opinion in Plant Biol. 11(3):278-285). Several metabolites from the pathway of lignin biosynthesis, including sinapyl alcohol, coniferon, caffeol glucose, acetovanillone and vanillic acid glucoside were significantly increased in the PheA* transgenic plants compared to control plants.

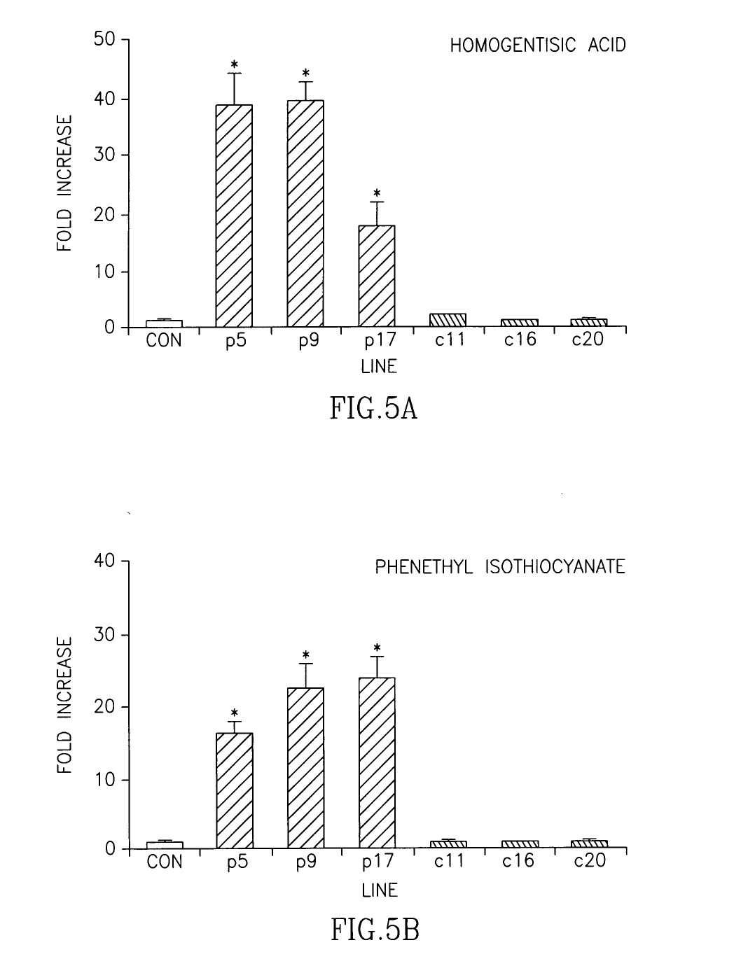

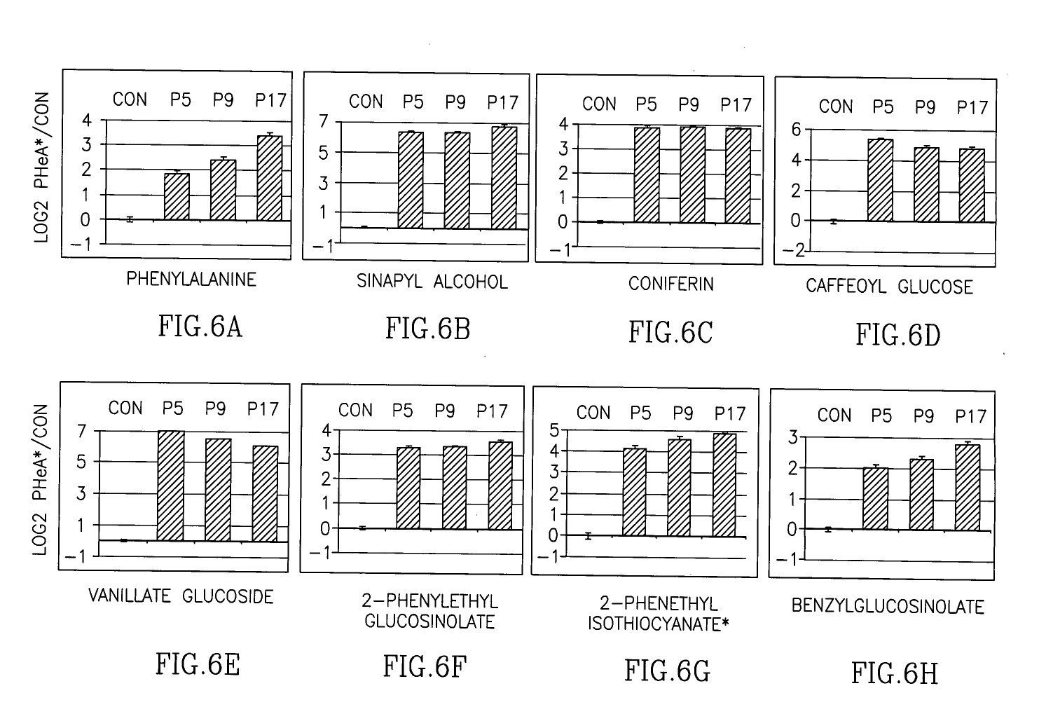

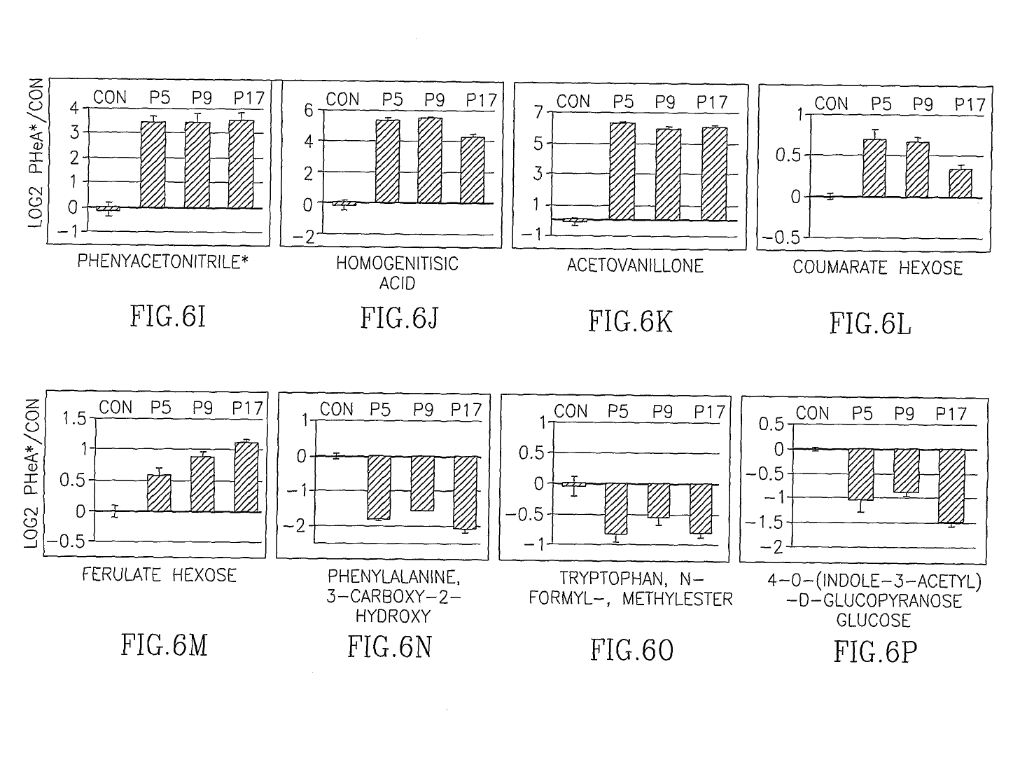

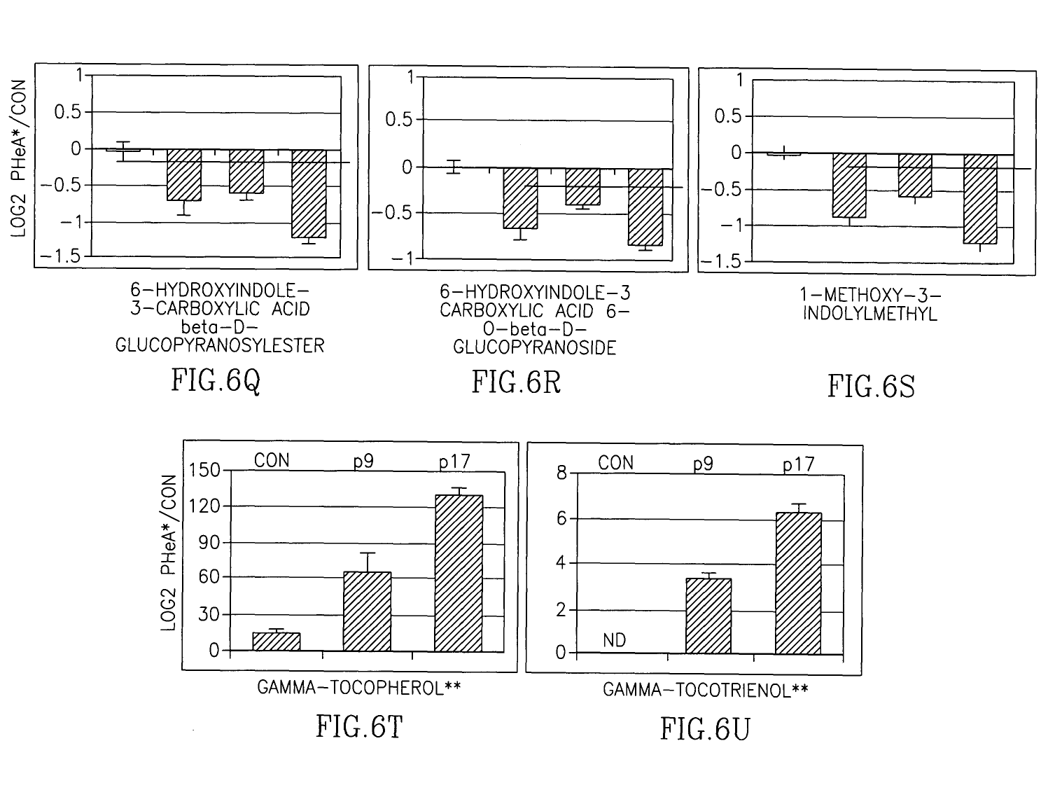

In addition, the levels of two glucosinolate derived from phenylalanine were significantly increased in the PheA* transgenic plants compared to control plants. These included 2-phenylethyl glucosinolate and benzyl glucosinolate (FIG. 6, f and h) as well as their derivatives: 2-phenylethyl isothiocyanate (PEITC) (FIG. 5A) and phenylacetonitrile (FIG. 6i). 2-Phenylethyl glucosinolate is hydrolyzed into the bioactive PEITC by the enzyme myrosinase, which is released from separate cellular compartments only when cells are damaged either by chewing or through food preparation (Barillari et al 2001 Fitoterapia. 72(7):760-764). It has been shown previously that root concentrations of 2-phenylethyl glucosinolate in Brassica napus influence the susceptibility of the crop to the root lesion nematode (Pratylenchus neglectus). In addition, this compound has a nematicidal effect on nematodes present on root tissues, with plants containing high levels of 2-phenylethyl glucosinolate shown to cause reduction in soil populations of P. neglectus (Potter et al 2000 Vol. 26(8):1811-1820).

PEITC was also indicated as one of the most effective cancer chemopreventive agents that could favorably modify the metabolism of carcinogens by inhibiting their Phase I enzyme-mediated activation and by inducing carcinogen-detoxifying Phase II enzymes (Huang et al 1998 Cancer Research 58(18):4102-4106). Moreover, the ability of PEITC to induce apoptosis by several mediators (depending on cell type) suggests that it may have an important therapeutic function, mainly in cases of resistance to chemotherapy due to mutation of p53 (Xu and Thornally 2000 Biochemical Pharmacology 60(2):221-231). It should be noted, however, that in spite of its anticancer activity, PEITC has been shown to be a strong promoter of urinary bladder carcinogenesis and to induce genotoxic effects and Cu(II)-mediated DNA damage (Paolini and Legator 1992 Nature 357(448) doi:10.1038/357448a).

Benzyl glucosinolate biosynthesis has been shown to involve the conversion of phenylalanine to phenylacetaldoxime catalyzed cytochrome P450 CYP79A2 from Arabidopsis (Wittstock and Halkier 2000 J Biol Chem 2000 275:14659-14666). Aromatic benzyl glucosinolate and myrosinase were found to be produced by suspended cells derived from hairy roots of Tropaeolum majus (Alvarez et al 2008 Plant Cell Physiol 49: 324-333). Phenylacetonitrile (also named benzyl cyanide) is a nitrile compound that is important in plant defense against pathogens (Fatouros at el 2008 PNAS105(29):10033-10038).

Unexpectedly, several compounds known as tyrosine catabolic products were also significantly increased in the PheA* transgenic plants compared to control plants: homogentistic acid, gamma-tocopherol and a gamma-tocotrienol. Homogentistic acid is the aromatic precursor of plasquinone and vitamin E (tocopherol and tocotrienol, Rippert et al 2004 Plant Physiol 134:92-100).

It is also shown by the present invention that expression of the PheA* proteins within the plant cell results in reduced tryptophan biosynthesis. In addition to its primary role as a building block for protein synthesis, tryptophan is a precursor for the synthesis of a large array of secondary metabolites, including the phytohormone Indole-3-acetic acid (IAA), antimicrobial phytoalexins, and indole glucosinolates that influence plant-pathogen interactions. Some tryptophan-derived glucosinolates are also known to be nutritionally harmful to human (Bell M J 1984 Journal of animal science 58:996-1010). Glucosinolates ((β-thioglucoside-N-hydroxysulfates; GSs) are nitrogen- and sulfur-containing plant specialized metabolites. Approximately 120 different GSs have been described up to date, almost all of them in the Brassicaceae family, with a common example of cruciferous crops, such as rapeseed, cabbage and broccoli. The occurrence of GSs in Arabidopsis promoted extensive studies on GSs biosynthesis, degradation and pathway regulation upon herbivore and other stress conditions. In Arabidopsis, there are at least 37 different GSs, with side chains derived mainly from methionine (Aliphatic Glucosinolates; AGs) and tryptophan (Indole Glucosinolates; IGs). The phytoalexins are antifungal chemicals made by plants in response to fungal attack and are considered by many to be involved in plant/fungal interactions. The toxic effects of GSs and their derivatives to animals nutrition is widespread (Bell M J 1984 ibid). Lowering GSs levels was essential for the development of central crops, such as rapeseed and Mustard (Brassica juncea) (Newkirk R W et al., 1997 Poult Sci 1997 76(9):1272-7). The nutritional advantages of low glocosinolate rapeseed meal, over the older type high glocosinolate meal, are clearly evident from feeding trials (Bell, M. J. 1984 ibid). The present invention now shows that 1-methoxy-3-indolylmethyl, a tryptophan GS, was decreased (FIG. 6 panel s). Furthermore, other tryptophan derivates were decreased: the indolic compound 4-O-(indole-3-acetyl)-D-glucopyranose (FIG. 6 panel p), tryptophan N-formyl-methyl ester (FIG. 6 panel o), 6-hydroxyindole-3-carboxylic acid 6-O-beta-glucopyranoside (FIG. 6 panel r) and 6-hydroxyindole-3-carboxylic acid beta-glucopyranosyl ester (FIG. 6 panel q).

In summary, the present invention shows for the first time that (i) production of the mutated E. coli PheA* either fused or not fused to a plastid transit peptide and hence localized either in the cytosol or plastid of a plant cell results in increased biosynthesis of phenylalanine and optionally of tyrosine, and reduced biosynthesis of tryptophan in the cell; (ii) E. coli PheA* expression in the plant cell lead to overproduction of tyrosine catabolic products; (iii) the synthesis of phenylalanine in plants may involve the activity of both chorismate mutase and prephenate dehydretase; (iv) elevated amounts of the precursor molecule phenylalanine in the plant cell leads to the production of elevated amounts of phenylalanine secondary metabolites within said cell; and (v) the synthesis of secondary metabolites derived from phenylalanine occurs mostly in plastids.

Producing the Transgenic Plants

Cloning of a polynucleotide encoding the PheA* polypeptide can be performed by any method as is known to a person skilled in the art. Various DNA constructs may be used to express PheA* in a desired plant.

The present invention provides a DNA construct or an expression vector comprising a polynucleotide encoding PheA*, which may further comprise regulatory elements, including, but not limited to, a promoter, an enhancer, and a termination signal.

Among the most commonly used promoters are the nopaline synthase (NOS) promoter (Ebert et al., 1987 Proc. Natl. Acad. Sci. U.S.A. 84:5745-5749), the octapine synthase (OCS) promoter, caulimovirus promoters such as the cauliflower mosaic virus (CaMV) 19S promoter (Lawton et al., 1987 Plant Mol. Biol. 9:315-324), the CaMV 35S promoter (Odell et al., 1985 Nature 313:810-812), and the figwort mosaic virus 35S promoter, the light inducible promoter from the small subunit of rubisco, the Adh promoter (Walker et al., 1987 Proc. Natl. Acad. Sci. U.S.A. 84:6624-66280, the sucrose synthase promoter (Yang et al., 1990 Proc. Natl. Acad. Sci. U.S.A. 87:4144-4148), the R gene complex promoter (Chandler et al., 1989 Plant Cell 1:1175-1183), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al. 1982 Cell 29:1015-1026). A plethora of promoters is described in International Patent Application Publication No. WO 00/18963.

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht 1 L et al. (1989 Plant Cell 1:671-680).

In particular embodiments of the present invention, the following elements were used to assemble the DNA constructs of the present invention:

1. A DNA sequence containing a cauliflower mosaic virus (CaMV) 35S promoter plus a CaMV omega translation enhancer upstream the translational initiation ATG codon, containing restriction enzyme sequences, termed 35S:PRO-Ω, SEQ ID NO:7 (Shaul 0 and Galili G 1993 Plant Mol Biol 23:759-768).

2. A DNA sequence containing the 3′ transcription termination and polyadenylation signals from the octopine synthase gene of Agrobacterium tumefacience, termed OCS-TER, with restriction enzyme sequences, SEQ ID NO:8 (Shaul 0 and Galili D 1993, ibid).

The above described sequences, SEQ ID NO: 7 and SEQ ID NO:8, are used as regulatory elements that enable the expression of the encoding nucleic acid sequence within a plant cell.

3. A DNA sequence encoding a pea rbcS3 plastid transit peptide, including the necessary restriction enzyme sequences (Shaul O and Galili D 1993, ibid, SEQ ID NO:9). The encoded peptide, when linked to the PheA* polypeptide, caused the migration of the later into the plastid.

4. A DNA sequence containing nucleotides 1 to 900 of the E. coli PheA gene (SEQ ID NO:10) which encode amino acids 1-300 of the E. coli CM/PDT enzyme (PheA* polynucleotide). The protein comprising these amino acids possesses the bifunctional CM and PDT activities, but each or both of these activities is less sensitive to feedback inhibition by phenylalanine compared to the analogous CM and/or PDT activities encoded by the wild type E. coli CM/PDT polypeptide, which contains the entire 386 amino acids encoded by the wild type E. coli PheA gene.

5. A DNA sequence encoding three copies of a hemagglutinin (HA) epitope tag (SEQ ID NO: 11). This epitope allows the detection of the recombinant PheA* polypeptide by immunoblots with antibodies for the HA epitope tag (Shevtsova et al., 2006 Eur J Neurosci 23:1961-1969).

According to particular embodiments, the polynucleotide comprises a nucleic acid sequence as set forth in SEQ ID NO:3. According to other particular embodiments, the encoded polypeptide, containing the pea rbcS3 plastid transit peptide and residues 1-300 of the E. coli PheA protein, has an amino acid sequence as set forth in SEQ ID NO:4.

Those skilled in the art will appreciate that the various components of the nucleic acid sequences and the transformation vectors described in the present invention are operatively linked, so as to result in expression of said nucleic acid or nucleic acid fragment. Techniques for operatively linking the components of the constructs and vectors of the present invention are well known to those skilled in the art. Such techniques include the use of linkers, such as synthetic linkers, for example including one or more restriction enzyme sites.

According to certain currently preferred embodiments, the DNA construct of the present invention comprises a nucleic acid sequence as set for the in SEQ ID NO:5. This DNA construct comprises a nucleic acid sequence encoding a 35S CaMV promoter and enhancer; a pea rbcS3 plastid transit peptide; a PheA* polypeptide; three copies of an hemagglutinin (HA) epitope tag and a nucleic acid sequence containing the 3′ transcription termination and polyadenylation signals from the octopine synthase gene of Agrobacterium tumefacience.

This construct encodes a polypeptide containing the pea rbcS3 plastid transit peptide, residues 1-300 of the E. coli PheA protein and three repeats of the HA epitope tag, having sequence ID NO:6.

According to yet another aspect, the present invention provides a method of altering the synthesis of at least one aromatic amino acid in a plant, comprising (a) transforming a plant cell with an exogenous polynucleotide encoding chorismate mutase and an exogenous polynucleotide encoding prephenate dehydratase; and (b) regenerating the transformed cell into a transgenic plant comprising at least one cell having an altered content of at least one aromatic acid compared to a corresponding cell of non transgenic plant.

Methods for transforming a plant cell with nucleic acids sequences according to the present invention are known in the art. As used herein the term “transformation” or “transforming” describes a process by which a foreign DNA, such as a DNA construct, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to preferred embodiments the nucleic acid sequence of the present invention is stably transformed into a plant cell.

There are various methods of introducing foreign genes into both monocotyledonous and dicotyledonous plants (Potrykus 11991 Annu Rev Plant Physiol Plant Mol Biol 42:205-225; Shimamoto K. et al., 1989. Nature 338:274-276).

The principal methods of the stable integration of exogenous DNA into plant genomic DNA include two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). A supplementary approach employs the Agrobacterium delivery system in combination with vacuum infiltration. The Agrobacterium system is especially useful in the generation of transgenic dicotyledenous plants.

Direct DNA uptake: There are various methods of direct DNA transfer into plant cells. In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as magnesium sulfate crystals or tungsten particles, and the microprojectiles are physically accelerated into cells or plant tissues.

According to certain embodiments, transformation of the DNA constructs of the present invention into a plant cell is performed using Agrobacterium system.

The transgenic plant is then grown under conditions suitable for the expression of the recombinant DNA construct or constructs. Expression of the recombinant DNA construct or constructs alters the quantity of phenylalanine and phenylpropanoids of the transgenic plant compared to their quantity in a non transgenic plant.

The regeneration, development and cultivation of plants from single plant protoplast transformants or from various transformed explants is well known in the art (Weissbach and Weissbach, In.: Methods for Plant Molecular Biology, (Eds.), 1988 Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

Selection of transgenic plants transformed with a nucleic acid sequence of the present invention as to provide transgenic plants having altered amount of aromatic amino acids and secondary metabolites derived therefrom is performed employing standard methods of molecular genetic, known to a person of ordinary skill in the art. According to certain embodiments, the nucleic acid sequence further comprises a nucleic acid sequence encoding a product conferring resistance to antibiotic, and thus transgenic plants are selected according to their resistance to the antibiotic. According to other embodiments, the antibiotic serving as a selectable marker is one of the aminoglycoside group consisting of paromomycin and kanamycin. According to yet further embodiments, the nucleic acid sequence further comprises a polynucleotide encoding at least one copy of the hemagglutinin (HA) epitope tag, operably linked to the polynucleotide encoding PheA*. According to certain currently preferred embodiments, the nucleic acid sequence comprises a polynucleotide encoding three copies of the hemagglutinin (HA) epitope. Proteins are then extracted and transgenic plants are selected according to the protein extracts reacting with HA-epitope antibodies.

Extraction and detection of the metabolites synthesized by the transgenic plant cells can be performed by standard methods as are known to a person skilled in the art. According to certain embodiments, the metabolites of the present invention are extracted and analyzed by GC-MS as described by Mintz-Oron et al., 2008 (Plant Physiol 147(2):823-51), LC-MS and HPLC as described by Fraser et al. 2000 (Plant J 24(4):551-558).

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines, or pollen from plants of these important lines is used to pollinate regenerated plants. A transgenic plant of the present invention containing a desired polypeptide is cultivated using methods well known to one of skill in the art.

There is a variety of methods in the art for the regeneration of plants from plant tissue. The particular method of regeneration will depend on the starting plant tissue and the particular plant species to be regenerated.

Also within the scope of this invention are seeds or plant parts obtained from the transgenic plants. Plant parts include differentiated and undifferentiated tissues, including but not limited to, roots, stems, shoots, leaves, pollen, seeds, tumor tissue, and various forms of cells and culture such as single cells, protoplasts, embryos, and callus tissue. The plant tissue may be in plant or in organ, tissue or cell culture.

The following non-limiting examples hereinbelow describe the means and methods for producing the transgenic plants of the present invention. Unless stated otherwise in the Examples, all recombinant DNA and RNA techniques, as well as horticultural methods, are carried out according to standard protocols as known to a person with an ordinary skill in the art.

### EXAMPLES

The present invention is further defined in the following Examples. These examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### MATERIAL AND METHODS

### Nucleic Acids Sequences

The following nucleic acid sequences were used in the production of DNA constructs of the present invention:

DNA sequence of the native E. coli PheA gene comprising 1161 bp; NCBI accession number AE014075, SEQ ID NO:2.
DNA sequence encoding the TP-PheA* polypeptide comprising a polynucleotide encoding the pea rbcS3 plastid transit peptide and a polynucleotide encoding residues 1-300 of the E. coli PheA polypeptide (SEQ ID NO:3, the nucleic acids encoding the PheA polypeptide sequence is in bold):

GTCGACTAGAAAATGGCTTCTATGATATCCTCTTCAGCTGTGACTACAGT
CAGCCGTGCTTCTACGGTGCAATCGGCCGCGGTGGCTCCATTCGGCGGCC
TCAAATCCATGACTGGATTCCCAGTTAAGAAGGTCAACACTGACATTACT
TCCATTACAAGCAATGGTGGAAGAGTAAAGTGCATGCCATCGGAAAACCC
GTTACTGGCGCTGCGAGAGAAAATCAGCGCGCTGGATGAAAAATTATTAG
CGTTACTGGCAGAACGGCGCGAACTGGCCGTCGAGGTGGGAAAAGCCAAA
CTGCTCTCGCATCGCCCGGTACGTGATATTGATCGTGAACGCGATTTGCT
GGAAAGATTAATTACGCTCGGTAAAGCGCACCATCTGGACGCCCATTACA
TTACTCGCCTGTTCCAGCTCATCATTGAAGATTCCGTATTAACTCAGCAG
GCTTTGCTCCAACAACATCTCAATAAAATTAATCCGCACTCAGCACGCAT
CGCTTTTCTCGGCCCCAAAGGTTCTTATTCCCATCTTGCGGCGCGCCAGT
ATGCTGCCCGTCACTTTGAGCAATTCATTGAAAGTGGCTGCGCCAAATTT
GCCGATATTTTTAATCAGGTGGAAACCGGCCAGGCCGACTATGCCGTCGT
ACCGATTGAAAATACCAGCTCCGGTGCCATAAACGACGTTTACGATCTGC
TGCAACATACCAGCTTGTCGATTGTTGGCGAGATGACGTTAACTATCGAC
CATTGTTTGTTGGTCTCCGGCACTACTGATTTATCCGCCATCAATACGGT
CTACAGCCATCCGCAGCCATTCCAGCAATGCAGCAAATTCCTTAATCGTT
ATCCGCACTGGAAGATTGAATATACCGAAAGTACGTCTGCGGCAATGGAA
AAGGTTGCACAGGCAAAATCACCGCATGTTGCTGCGTTGGGAAGCGAAGC
TGGCGGCACTTTGTACGGTTTGCAGGTACTGGAGCGTATTGAAGCGAATC
AGCGACAAAACTTCACCCGATTTGTGGTGTTGGCACGTAAAGCCATTAAC
GTTTCTGACCAGGTTCCGGCGAAAACGACGTTGTAAGAATTC

DNA sequence encoding the 35S-PRO-Ω-TP-PheA*-3HA polypeptide comprising a polynucleotide encoding the pea rbcS3 plastid transit peptide, a polynucleotide encoding residues 1-300 of the E. coli PheA polypeptide, and a polynucleotide encoding three repeats of the HA epitope tag and OCS— transcription terminator (SEQ ID NO:5, the PheA polypeptide sequence is bolded):

GGTACCCGGGGATCCCCCCTCAGAAGACCAGAGGGCTATTGAGACTTTTC
AACAAAGGGTAATATCGGGAAACCTCCTCGGATTCCATTGCCCAGCTATC
TGTCACTTCATCGAAAGGACAGTAGAAAAGGAAGGTGGCTCCTACAAATG
CCATCATTGCGATAAAGGAAAGGCTATCGTTCAAGATGCCTCTACCGACA
GTGGTCCCAAAGATGGACCCCCACCCACGAGGAACATCGTGGAAAAAGAA
GACGTTCCAACCACGTCTTCAAAGCAAGTGGATTGATGTGATATCTCCAC
TGACGTAAGGGATGACGCACAATCCCACTATCCTTCGCAAGACCCTTCCT
CTATATAAGGAAGTTCATTTCATTTGGAGAGGACAGGCTTCTTGAGATCC
TTCAACAATTACCAACAACAACAAACAACAAACAACATTACAATTACTAT
TTACAATTACAGTCGACTAGAAAATGGCTTCTATGATATCCTCTTCAGCT
GTGACTACAGTCAGCCGTGCTTCTACGGTGCAATCGGCCGCGGTGGCTCC
ATTCGGCGGCCTCAAATCCATGACTGGATTCCCAGTTAAGAAGGTCAACA
CTGACATTACTTCCATTACAAGCAATGGTGGAAGAGTAAAGTGCATGCCA
TCGGAAAACCCGTTACTGGCGCTGCGAGAGAAAATCAGCGCGCTGGATGA
AAAATTATTAGCGTTACTGGCAGAACGGCGCGAACTGGCCGTCGAGGTGG
GAAAAGCCAAACTGCTCTCGCATCGCCCGGTACGTGATATTGATCGTGAA
CGCGATTTGCTGGAAAGATTAATTACGCTCGGTAAAGCGCACCATCTGGA
CGCCCATTACATTACTCGCCTGTTCCAGCTCATCATTGAAGATTCCGTAT
TAACTCAGCAGGCTTTGCTCCAACAACATCTCAATAAAATTAATCCGCAC
TCAGCACGCATCGCTTTTCTCGGCCCCAAAGGTTCTTATTCCCATCTTGC
GGCGCGCCAGTATGCTGCCCGTCACTTTGAGCAATTCATTGAAAGTGGCT
GCGCCAAATTTGCCGATATTTTTAATCAGGTGGAAACCGGCCAGGCCGAC
TATGCCGTCGTACCGATTGAAAATACCAGCTCCGGTGCCATAAACGACGT
TTACGATCTGCTGCAACATACCAGCTTGTCGATTGTTGGCGAGATGACGT
TAACTATCGACCATTGTTTGTTGGTCTCCGGCACTACTGATTTATCCGCC
ATCAATACGGTCTACAGCCATCCGCAGCCATTCCAGCAATGCAGCAAATT
CCTTAATCGTTATCCGCACTGGAAGATTGAATATACCGAAAGTACGTCTG
CGGCAATGGAAAAGGTTGCACAGGCAAAATCACCGCATGTTGCTGCGTTG
GGAAGCGAAGCTGGCGGCACTTTGTACGGTTTGCAGGTACTGGAGCGTAT
TGAAGCGAATCAGCGACAAAACTTCACCCGATTTGTGGTGTTGGCACGTA
AAGCCATTAACGTTTCTGACCAGGTTCCGGCGAAAACGACGTTGGAATTC
ATCTTTTACCCATACGATGTTCCTGACTATGCGGGCTATCCCTATGACGT
CCCGGACTATGCAGGATCCTATCCATATGACGTTCCAGATTACGCTGCTC
AGTAGTCTAGAGTCCTGCTTTAATGAGATATGCGAGACGCCTATGATCGC
ATGATATTTGCTTTCAATTCTGTTGTGCACGTTGTAAAAAACCTGAGCAT
GTGTAGCTCAGATCCTTACCGCCGGTTTCGGTTCATTCTAATGAATATAT
CACCCGTTACTATCGTATTTTTATGAATAATATTCTCCGTTCAATTTACT
GATTGTACCCTACTACTTATATGTACAATATTAAAATGAAAACAATATAT
TGTGCTGAATAGGTTTATAGCGACATCTATGATAGAGCGCCACAATAACA
AACAATTGCGTTTTATTATTACAAATCCAATTTTAAAAAAAGCGGCAGAA
CCGGTCAAACCTAAAAGACTGATTACATAAATCTTATTCAAATTTCAAAA
GGCCCCAGGGGCTAGTATCTACGACACACCGAGCGGCGAACTAATAACGT
TCACTGAAGGGAACTCCGGTTCCCCGCCGGCGCGCATGGGTGAGATTCCT
TGAAGTTGAGTATTGGCCGTCCGCTCTACCGAAAGTTACGGGCACCATTC
AACCCGGTCCAGCACGGCGGCCGGGTAACCGACTTGCTGCCCCGAGAATT
ATGCAGCATTTTTTTGGTGTATGTGGGCCCCAAATGAAGTGCAGGTCAAA
CCTTGACAGTGACGACAAATCGTTGGGCGGGTCCAGGGCGAATTTTGCGA
CAACATGTCGAGGCTCAGCAGGACCTGCAGGCATGCAAGCTAGCTTACTA
GT

35S:PRO-Ω, the Cauliflower 35S constitutive promoter with Ω translation enhancer, SEQ ID NO:7.
DNA encoding pea rbcS3 plastid transit peptide, having SEQ ID NO:9.
PheA*-Nucleic acids 1-900 (SEQ ID NO: 10) of the native E. coli PheA gene (1161 bp; NCBI accession number AE014075, SEQ ID NO:2).
PheA* with restriction enzyme sequences, SEQ ID NO:12 comprising ‘5 GCATGC and 3’ GAATTC restrictions enzyme sequences,
PheA* with restriction enzyme sequences, SEQ ID NO:13 comprising ‘5 AAGCTT and 3’ GAATTC restriction enzyme sequences.
DNA encoding HA epitope tag-three repeats including sites for restriction enzymes, (SEQ ID NO:11).

OCS— transcription terminator with ‘5 TCTAGA and 3’ ACTAGT restriction enzyme sequences (SEQ ID NO:8).

### Recombinant Genes

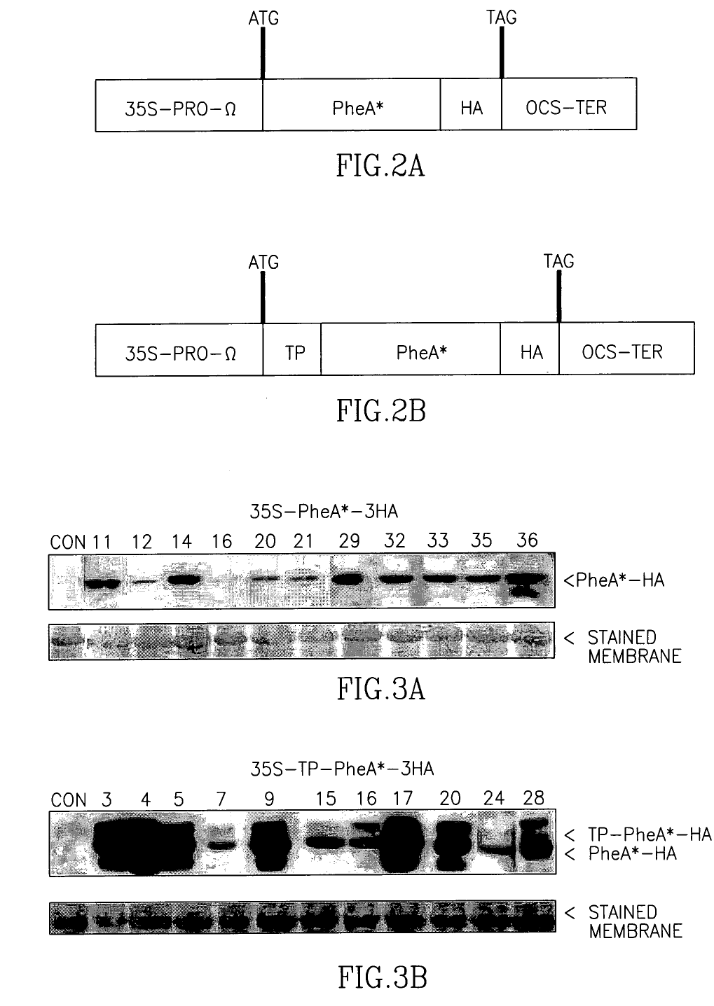

Two kinds of recombinant genes were constructed aiming to target the recombinant PheA* polypeptide either into the cytosol (FIG. 2; construct A) or to the plastid (FIG. 2; construct B) inside plant cells, each of which containing the HA epitope tag. These recombinant genes were termed as 35S:PRO-PheA*-HA and 35S:PRO-TP-PheA*-HA, respectively.

### Plasmid Construction

The truncated coding DNA sequence of the E. coli PheA gene, encoding the CM and PDT domains, was amplified by PCR with the following primers: 5′-GCCAAGCTTATGGGCATGCCATCGGAAAACCCGTTACTGGC-3′ (SEQ ID NO:14) that introduces an SphI restriction site (underlined); and 5′-CCCCGGAATTC CAACGTCGTTTTCGCCGGAACCTG-3′ (SEQ ID NO:15) that introduces an EcoRI restriction site (underlined). The PCR product was fused in frame at its 5′ end to a DNA encoding RUBISCO small subunit-3A plastid transit peptide The PCR product was fused in frame at its 5′ end to a DNA encoding RUBISCO small subunit-3A plastid transit peptide fused at its 5′ end to a 35S promoter (Shaul and Galili 1993 ibid) and at its 3′ to a DNA encoding three copies of a HA epitope tag fused to an octopine synthase terminator, and the this chimeric gene was sub-cloned into the Ti Plasmid pART27 (Gleave AP 1992 Plant Mol Biol 20, 1203-1207). Both strands of the PheA DNA in pART27 were sequenced to ensure that no undesired mutations have been introduced during the course of the PCR amplification. The chimeric PheA* gene was introduced into Agrobacterium tumefacies strain EHA-105 and used to transform Arabidopsis plants as previously described (Clough S J and Bent A F 1998 Plant J., 16, 735-743).

### Arabidopsis Stable Floral Transformation

Wild type (Wt) Arabidopsis thaliana ecotype Colombia plants were inoculated by submersing inflorescences in the transformed A. tumefacies culture as previously described (Clough S J and Bent A F, 1998 Plant J 16, 735-743).

### Plant Material and Growth Condition

Seeds were collected, dried, sterilized (Slavikova S et al., 2005 J Exp Bot 56, 2839-2849) and sow on Petri dishes containing Nitsch complete medium pH 6 (Duchefa, Haarlem, Netherlands) supplemented with 1 or 2% sucrose and 1% plant agar. For transgenic plants selection, 50 μg/ml kanamycin was added to the growth medium (Clough S J and Bent A F 1998, ibid). The seeds were imbibed for 48 h at 4° C. and transferred to a climate-controlled growth room with a regime of 16h light/8h dark (long day conditions) at 22° C. The resistant seedlings were removed to soil and grown in the greenhouse at 22° C. under long day conditions.

### Selection of Transgenic Lines

Western blot analysis was performed with anti-HA tag antibodies, in order to identify the mutated plants which are translated the chemeric gene (Stepansky A and Galili G 2003 Plant Physiol 133, 1407-1415). Additionally, T₂ generation plants were examined and lines with a single insertion gene were selected based on 3:1 genetic segregation. For testing the growth of the transgenic plants producing elevated concentrations of the compounds of the invention, homozygous PheA* plants were germinated on regular Nitch medium. The 4-Hydroxy-phenylpyruvate dioxygenase (HPPD) inhibitor Isoxaflutole was used as previously described (Rippert P et al. 2004 Plant Physiol., 134:92-100).

### Tocopherol and Tocotrienol Extraction and Analysis

Tocopherol and tocotrienol extraction was performed essentially as previously described (Bino et al., 2005 New Physiol 166:427-438; Fraser et al., 2000 Plant J 24(4):551-558) with the following modifications: Frozen Arabidopsis shoot powder (100 mg) was extracted with 0.5 ml Methanol containing 0.1% butylated hydroxytoluene (BHT). The samples were shaken for 5 min at 4° C. and then 0.5 ml of 50 mM tris-HCl plus 1M NaCl, pH 7.5 was added and the samples were shaken for 10 min at 4° C. Then, 0.4 ml of cold chloroform was added and the samples were shaken for 10 min at 4° C., and then centrifuged at 10,000 rpm, 4° C. for 10 min. The supernatant was collected into a new tube and re-extracted with 0.2 ml cold chloroform and the samples were shaken for 10 min at 4° C. and centrifuged at 10,000 rpm, 4° C. for 10 min. The chloroform extract fractions were combined, dried under a stream of nitrogen gas and re-suspended in 100 μl ethylacetate. Extracts were shielded from strong light during the entire preparation

HPLC system was consisted from the Waters 2690 separation module (Waters Chromatography, Milford, Mass., USA) and the Waters 2996 Photo Diode Array Detector. YMC-Pack reverse-phase C30 column (250×4.6 mm; 5 μm), coupled to a 4×3 mm C18 guard (Phenomenex) and maintained at 30° C. was used for the compounds separation. The mobile phase composition, the gradient and the flow rate was as described by Fraser et al. (2000, ibid). The UV spectra were monitored from 200 nm till 750 nm. Data were collected and analyzed using the Waters Millennium32 software. Absorbance spectra and retention times of eluting peaks were compared with those of commercially available standards:

δ-tocopherol, γ-tocopherol (Supelco), α-tocopherol (Aldrich), α-tocotrienol, γ-tocotrienol and δ-tocotrienol (Cayman Chemical Company) were putatively identified by comparison of their absorbance spectra and retention times to the data presented by Fraser et al. (2000, ibid). Peak areas of the compounds were determined at the wavelength providing maximum absorbance using the Waters Millennium32 software supplied.

### Non-Targeted Metabolic Analysis of Semi-Polar Compounds by HPLC-qTOF-MS

Non-targeted metabolic analysis was performed on shoot material of plants overexpressing the PheA* (n=6) and wild-type (con n=6). Arabidopsis shoots were collected from 10 days olds plants (100 mg), frozen, grinded and extracted. Extraction was performed in 450 μl of MeOH:H₂O (80:20). Plant extract was sonicated for 30 min and centrifuged for 5 min at 10,000g. After centrifugation, the supernatant was filtered through a Millex-GV MF (PDV) 0.22 μm filter and analyzed by liquid chromatography—mass spectrometry (LC-MS). The sample (5 μl) was applied to an HPLC-qTOF instrument (Waters Premier QTOF, Milford, Mass., USA), with the HPLC column connected on-line to a UV detector. Samples were separated on a BEH C18 Acquity column (100×2.1-mm, 1.7 μm; Waters) under a linear gradient elution program with solvent A (0.1% formic acid in 5% acetonitrile/95% water) and solvent B (0.1% formic acid in acetonitrile): 0 to 28% solvent B (22 min), 28 to 40% solvent B (till 22.5 min), 40 to 100% solvent B (till 23 min), 100% solvent B (till 24.5 min), and 100% solvent A (till 26 min). Elution was operated at 0.3 ml/min flow and the column temperature was set at 35° C. Sample set was performed both as positive and negative ionization modes. The electrospray probe was operated at 3 kV. The source and desolvation temperatures were 125° C. and 275° C., respectively. A mixture of 15 standard compounds, injected after each set of 10 samples was used as the quality control sample. The MassLynx software version 4.1 (Waters) was used to control the instrument and calculate accurate masses.

### Analysis of LC-qTOF-MS Metabolomics Data

The analysis of the raw HPLC-QTOF-MS data was performed using the XCMS software (Smith et al., 2006 Anal Chem 78:779-787) from the Bioconductor package (v. 2.1) for R statistical language (v. 2.6.1). XCMS performs chromatogram alignment, mass signal detection and peak integration. The XCMS set was constructed with the following parameters: fwhm=10.8, step=0.05, steps=4, mzdiff=0.07, snthresh=8, max=1000. Since injections of samples in the positive and negative ionization modes were performed in the separate injection sets, XCMS pre-processing was done for each ionization mode independently. Putatively assigned metabolites, analyzed using the HPLC-QTOF-MS (60 metabolites) were statistically treated using t-test, employing JMP software. Metabolites were identified using standard compounds by comparison of their retention times, UV spectra, MS/MS fragments and dual energy fragments. Identification of metabolites for which standards were not available was carried out as described by Malitsky et al. (2008 Plant Physiol Published on line Oct. 1, 2008; 10.1104/pp. 108.124784)

### GC-MS Profiling of Derivatized Extracts

The GC-MS analysis was performed on shoot extracts of plants overexpressing the PheA* (five to six replicates per genotype). Analysis of polar compound extracts was performed following the protocol described by Mintz-Oron et al., (2008, ibid). Briefly, frozen grinded tissue powder (100 mg) was extracted in 700 μl of methanol with 60 μl of internal standard (ribitol, 0.2 mg in 1 ml of water). After mixing vigorously, the extract was sonicated in a bath sonicator for 20 min., and centrifuged at 20,000 g. Chloroform (375 μl) and water (750 μl) were added to the supernatant and the mixture was vortexed and centrifuged. Aliquots of the upper methanol/water phase (500 μl) were taken and lyophilized. The derivatization method of the lyophilized sample was as described by Mintz-Oron et al. (2008 ibid). Sample volumes of 1 μl were injected into the GC column. A retention time standard mixture (14 μg/ml in pyridine: n-dodecane, n-pentadecane, n-nonadecane, n-docosane, n-octacosane, n-dotracontane, and n-hexatriacontane) was injected after each set of six samples. The GC-MS system was comprised of a COMBI PAL autosampler (CTC analytics AG), a Trace GC Ultra gas chromatograph equipped with a PTV injector, and a DSQ quadrupole mass spectrometer (ThermoElectron Cooperation, Austin, USA). GC was performed on a 30 m×0.25 mm×0.25 μm Zebron ZB-5 ms MS column (Phenomenex, USA). The PTV split technique was carried out as follows: samples were analyzed in the PTV solvent split mode. PTV inlet temperature was set at 45° C., followed by a temperature program: hold at 45° C. for 0.05 min, raise to 70° C. with a ramp rate of 10° C./sec, hold at this temperature for 0.25 min, transfer-to-column stage (raising to 270° C. with a ramp rate of 14.5° C./sec; hold at 270° C. for 0.8 min.), and finish by a cleaning stage (raising to 330° C. with a ramp rate of 10° C./sec; hold at 330° C. for 10 min). For separation of the metabolites we used the chromatographic GC conditions described at Mintz-Oron et al., (2008, ibid).

### Analysis of GC-MS Data

The reconstructed ion chromatograms and mass spectra were evaluated using the Xcalibur software v.1.4 (ThermoFinnigan, Manchester, UK). Compounds were identified by comparison of their retention index (RI) and mass spectrum to those generated for authentic standards analyzed on the same instrument. When the corresponding standards were not available, compounds were putatively identified by comparison of their RI and mass spectrum to those present in the mass spectra library of Max-Planck-Institute for Plant Physiology, Golm, Germany (Q_MSRI_ID, http://csbdb.mpimp-golm.mpg.de/csbdb/gmd/msri/gmd_msri.html) and the commercial mass spectra library NIST (www.nist.gov). The response values for metabolites resulting from the Xcalibur processing method were normalized to the Ribitol internal standard. This was carried out by dividing the peak area of the metabolite by the peak area of the internal standard.

### Statistical Analysis of GC-MS Data

In order to test if the level of each metabolite in the transgenic line was significantly different from its levels in the wild type plants, one way ANOVA employing TMEV4 software was used (Saeed et al., 2003 Biotechniques 34(2):374-8; Scholz et al, 2004 Bioinformatics 20(15):2447-54). T-test was performed for metabolites level using JMP software.

### EXAMPLE 1

### Expression of the Recombinant PheA* Genes in Transgenic Arabidopsis Plants

To test the metabolic significance of PPY in plants, Arabidopsis plants were transformed with a transgene containing the coding DNA sequence of a truncated bacterial PheA* gene fused to the cauliflower mosaic virus 35S promoter. This bacterial gene encodes a bifunctional Phe-insensitive CM/PDT PheA* enzyme that catalyzes the conversion of chorismate via prephenate into PPY. A DNA encoding a RUBISCO small subunit-3A plastid transit peptide was optionally fused in frame to 5′ end of the PheA* open reading frame to direct the bacterial enzyme into the organelle where the major pathway of phenylalanine takes place. The coding DNA sequence of the bacterial gene was also fused in frame on its 3′ end to a DNA encoding three copies of a hemagglutinin (HA) epitope tag, to enable the detection of the protein. This construct (SEQ ID NO:5) was transformed into Arabidopsis plants and plants with a single insertion were selected based on 3:1 genetic segregation in the T2 generation (the two recombinant 35S:PRO-PheA*-HA and 35S:PRO-TP-PheA*-HA genes are described in FIG. 2). To examine the polypeptides produced by these recombinant genes, an immunoblot assay with commercial monoclonal antibodies against the HA epitope tag was used. Independently transformed plants generated from transformation with the recombinant 35S:PRO-PheA*-HA gene lacking the DNA encoding the plastid transit peptide exhibited a single major polypeptide band, corresponding in size to the expected PheA*-HA polypeptide (FIG. 3A, band marked as PheA*-HA on the right). Independently transformed plants generated from transformation with the recombinant 35S:PRO-TP-PheA*-HA gene containing the DNA encoding the plastid transit peptide exhibited two major bands; the rapidly migrating band corresponding in size to the expected PheA*-HA polypeptide (FIG. 3B, marked as PheA*-HA on the right), while the slower band corresponded in size to the PheA*-HA plus the plastid transit peptide (marked as TP-PheA*-HA on the right). This indicates that a significant proportion of the TP-PheA*-HA polypeptide encoded by the plastidic PheA*-HA construct was translocated into the plastid, a process that causes the cleavage of the transit peptide to generate the mature PheA*-HA polypeptide. Next, T₂ generation progeny plants were produced from several independently transformed plants expressing either the 35S:PRO-PheA*-HA or the 35S:PRO-TP-PheA*-HA, and several individual genotypes segregating 3:1 for the kanamycin resistance gene were selected for further analyses. These genotypes are expected to contain the recombinant constructs inserted only into a single place in the Arabidopsis genome.

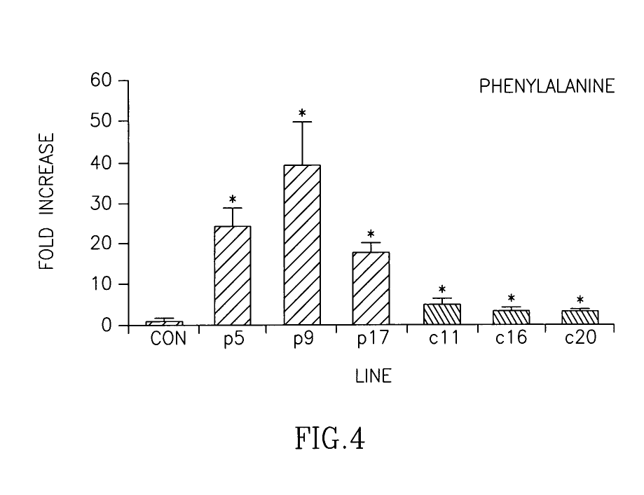

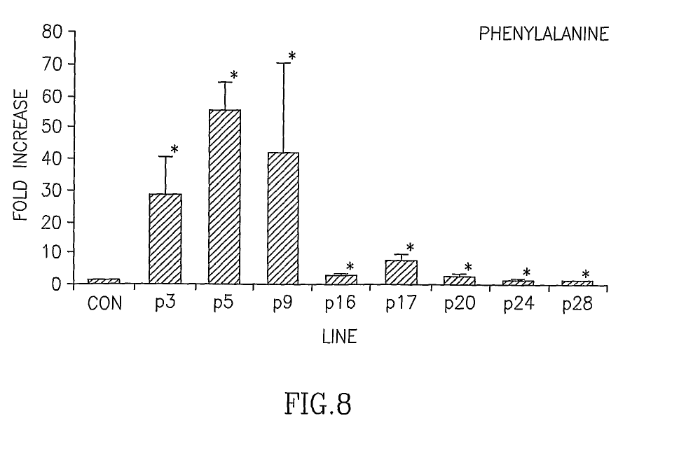

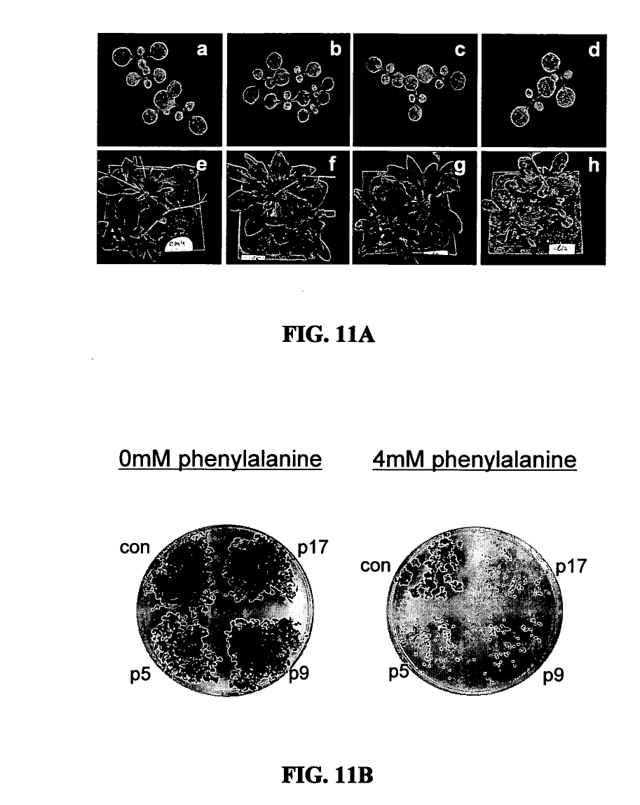

For a further initial test, aiming at verifying whether expression of the PheA* polypeptide alters the synthesis of Phe, rosette leaves of two month old plants derived from ten independently transformed genotypes (genotypes p3, p5, p9, p15, p16, p17, p20, p24, p28, p29; three replicates from each genotype) as well as from nine replicates of the control genotype expressing an empty Ti vector (each sample is derived from a pool of at least four individual plants) were subjected to a GC-MS analysis (total of 39 individual runs). As shown in FIG. 8, the individual transgenic genotypes exhibited various degrees of elevation in the levels of phenylalanine, reaching up to ˜50 higher Phe level than the average phenylalanine level in the control plants. Based on the immunoblot analysis and phenylalanine content of 10 days old plants (FIGS. 3 and 4), three representative independently transformed genotypes with moderate (p5) and relatively high (p9 and p17) expression level of the PheA* polypeptide for further analysis and those were used to generate homozygous lines. As shown in FIG. 11A, upon growth in soil, these three transgenic genotypes showed quite normal phenotype of seedlings (panels b-d) and more mature plants (panels f-h, plants shown in panels a and e being control plants) except for the genotype p17, which in some cases showed minor alteration in leaf structure (panel h).

Amino acids are generally toxic to plants when added externally at relatively high concentrations. We therefore also used this trait to further confirm that the PheA* plants overproduce phenylalanine, by testing for sensitivity to external application of phenylalanine. As shown in FIG. 11B, while the control and PheA* genotypes germinated with comparable phenotypes on media lacking phenylalanine (left plate), germination of the PheA* genotypes was significantly inhibited compared to the control genotype upon exposure to 4 mM phenylalanine.

### EXAMPLE 2

### Metabolic Profiling Analyses of Selected Transgenic Arabidopsis Plants

### Analyses of Phenylalanine and Secondary Metabolites

To study the effect of expression of the recombinant 35S:PRO-PheA*-HA and 35S:PRO-TP-PheA*-HA genes on the production of phenylalanine and PPY, leaves of 10 days old plants from several representative genotypes of the plants expressing each of the two genes were examined for their metabolic status by gas chromatography mass spectroscopy (GC-MS), liquid chromatography—mass spectrometry (LC-MS) and high pressure liquid chromatography (HPLC) analyses. These analyses can detect numerous metabolites. As shown in FIG. 4, both types of transgenic plants show elevated levels of phenylalanine compared to control plants transformed with empty vector. Yet phenylalanine level in plants expressing the plastidic 35S:PRO-TP-PheA*-HA* (lines p5, p9 and p17) had significantly higher levels of phenylalanine than plants expressing the cytosolic 35S:PRO-PheA*-HA (lines c11, c16 and c20).

At the next stage, the leaves from two-month-old plants were subjected to GC-MS analysis. As shown in FIG. 8, also at this developmental stage, the transgenic plants, expressing the plastidic 35S:PRO-TP-PheA*-HA gene and 35S:PRO-PheA*-HA gene accumulated significantly higher levels of phenylalanine (lines p3, p5, p9, p16, p17, p20, p24 and p28). Not only phenylalanine, but the levels of at least two metabolites, identified by commercially available standards as homogentistic acid and phenethyl isothiocyanate, were higher in the transgenic plants expressing the plastidic 35S:PRO-TP-PheA*-HA gene than in the control plants (FIG. 5 A, B; compare lines p5, p9 and p17 with the control plants). The levels of these two metabolites in plants expressing the cytosolic 35S:PRO-PheA*-HA gene were not significantly different from that of the control plants (FIG. 5A, B; compare lines c11, c16 and c20 with the control plants). The elevated level of homogentistic acid, which is a catabolic product of tyrosine, may be attributed to elevated biosynthesis of tyrosine by the transgenic plants.

Most of the elevated metabolites belong to the different networks downstream of phenylalanine, including lignin-associated metabolites (sinapyl alcohol or coniferon, or caffeol glucose, or acetovanillone or vanillic acid glucoside), phenylalanine glucosinolates and their derivates (phenethyl isothiocyanate, 2-phenylethyl glucosinolate, benzyl glucosinolate and phenylacetonitrile) (FIG. 6).

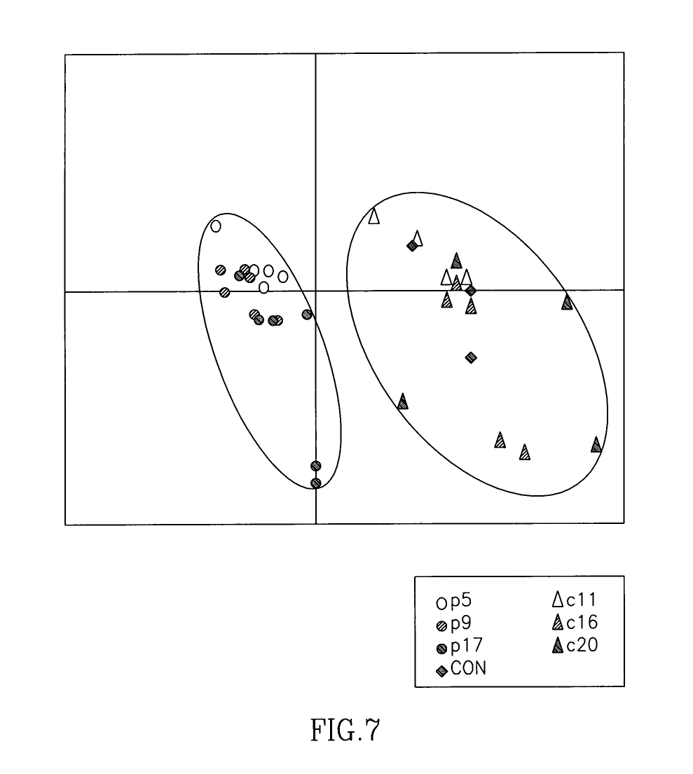

To get a global view on the metabolic changes the data were analyzed by principal component analysis (PCA; Saeed et al., ibid; Scholz et al., 2004, ibid). The PCA showed pronounce difference between transgenic plants expressing the plastidic 35S:PRO-TP-PheA*-HA gene (lines p5, p9 and p17) and those expressing the cytosolic 35S:PRO-PheA*-HA gene (lines c11, c16, c20), which were grouped together with the control line. This indicates that expression of the plastidic 35S:PRO-TP-PheA*-HA gene causes changes in the levels of a number of metabolites (FIG. 7).

### Catabolism of Tyrosine

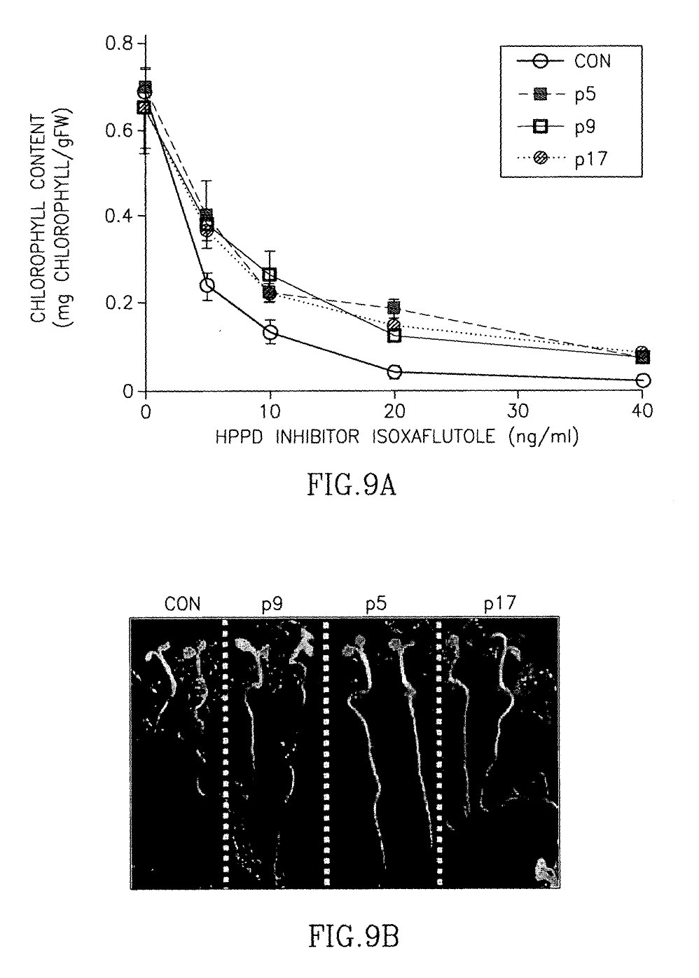

Tyrosine is catabolized in plants to a number of metabolites including hydroxyl-phenylpyruvate (p-HPP), homogentistic acid (HGA) and tocopherols. One of the enzymes in the Tyr catabolism pathways is 4-Hydroxy-phenylpyruvate dioxygenase (HPPD), which catalyzes the conversion of p-HPP to HGA. HGA is derived exclusively from the catabolism of tyrosine and is also a substrate for a number of other metabolites, such as tocopherols. Tocopherols are members of a large, multifunctional family, of lipid-soluble compounds called prenylquinones that also include tocotrienols, plastoquinones, and phylloquinones (vitamin K1). HPPD is also a specific target of several herbicide families, such as isoxazoles, triketones and pyroxazoles. Inhibition of its activity results in the depletion of the plant plastoquinone and tocopherol pools, leading to bleaching symptoms. These herbicides are very potent for the selective pre- and in some cases also post-emergence control of a wide range of broadleaf and grass weeds in maize and rice. Their herbicidal potential raises interest in the development of highly resistant transgenic crops. To test whether expression of the bacterial PheA* construct leads to enhanced tyrosine catabolism Isoxaflutole (Bayer CropScience), a pre- and early post-emergence herbicide, which inhibits HPPD activity was used. In addition to the results presented hereinabove showing high level of HGA in the transgenic plants expressing the 35S:PRO-TP-PheA*-3HA gene (FIG. 5), it was found that these transgenic plants were more resistant to Isoxaflutole, compared to control plants expressing an empty Ti plasmid. Control plants developed chlorotic symptoms in response to 10 ppb Isoxaflutole, while the transgenic plants remained green (FIG. 9).

We also tested the effect of PheA* expression on the production of catabolic products of Tyr. Notably, although we could not detect increased level of tyrosine in plants expressing PheA*, the level of its second downstream metabolite homogentistic acid (HGA) was significantly increased in the PheA* plants, compared to the control plants (FIG. 5A). Since homogentistic acid is a precursor for the vitamin E associated metabolites tocopherols and tocotrienols (DellaPenna and Pogson 2006), the levels of these metabolites, using HPLC fractionation was also measured. The levels of both, homogentistic acid (FIG. 5A) as well as a gamma-tocopherol and a gamma-tocotrienol (FIG. 6 panels o and p) were significantly higher in the PheA* plants, compared to the control plants.

### Biosynthesis of Tryptophan

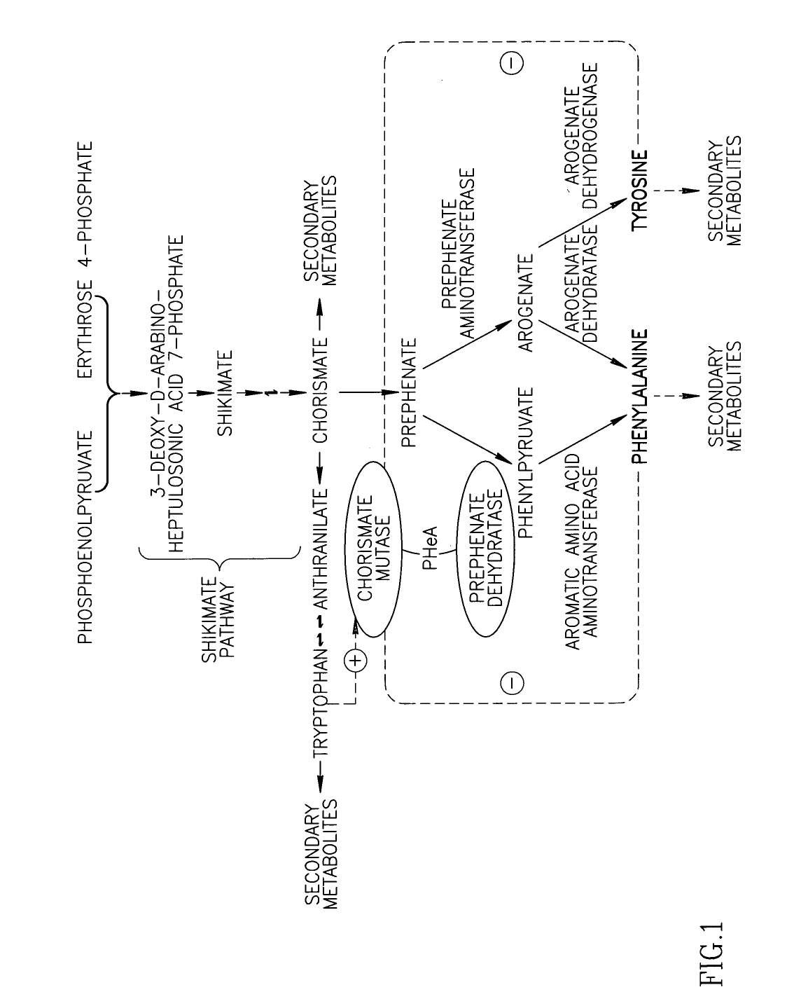

Since phenylalanine and tryptophan biosyntheses compete on their common precursor chorismate (FIG. 1), it was examined whether PheA* expression cross regulates the biosynthesis of tryptophan. Even though no significant change in Trp level between the PheA* and the control plants was observed, the level of a number of Trp catabolic products was lower in the PheA* that in the control plants, suggesting that PheA* expression enhanced the flux from chorismate to prephenate, competing down the flux towards Trp biosynthesis.

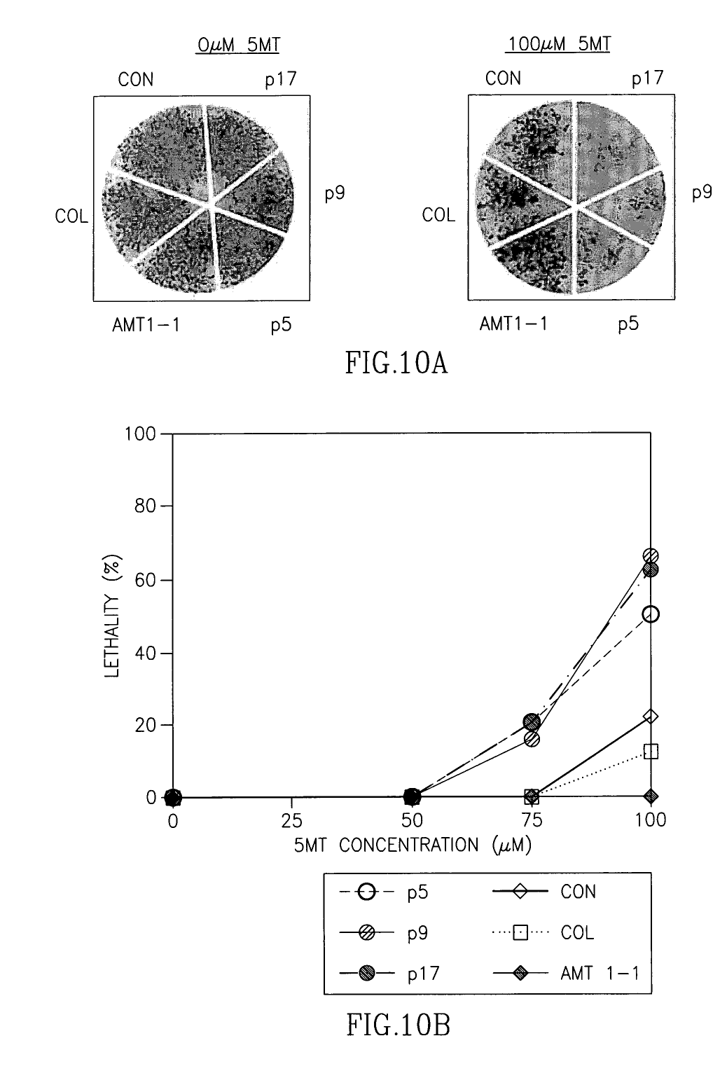

To evaluate the biosynthesis level of tryptophan, the effects of tryptophan analog on the transgenic plants of the present invention and on positive-control and control plants were examined. Plants used were as follows: i) plants of the present invention transformed with the 35S:PRO-TP-PheA*-3HA construct; (ii) positive-control transgenic plants having the dominant mutation amt1-1, which are resistant to tryptophan analogs due to high levels of tryptophan (5× normal) and anthranilate synthase (2× normal), the first enzyme of tryptophan biosynthesis; (iii) control plants expressing an empty Ti vector; and (iv) wild type Arabidopsis Colombia ecotype (col). Plants were grown for three weeks on zero and 100 μM 5-methyl tryptophan (5MT). As is shown in FIG. 10, the growth of plants p17, p9 and p5 expressing the 35S:PRO-TP-PheA*-3HA polynucleotide is strongly inhibited by 5MT, apparently due to reduced tryptophan biosynthesis.

Several catabolic products of tryptophan had a decreased level in the transgenic plants of the invention compared to the control plants. These catabolic products of tryptophan include 1-methoxy-3-indolylmethyl (FIG. 6 panel s), 6-hydroxyindole-3-carboxylic acid 6-O-beta-D-glucopyranoside (FIG. 6 panel r), 6-hydroxyindole-3-carboxylic acid beta-D-glucopyranosyl ester (FIG. 6 panel q), 4-O-(indole-3-acetyl)-D-glucopyranose (FIG. 6 panel p) and tryptophan N-formyl-methyl ester (FIG. 6 panel o).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-54. (canceled)

55. A transgenic plant comprising at least one plant cell comprising an exogenous polynucleotide encoding chorismate mutase (CM) and a polynucleotide encoding prephenate dehydratase (PDT), wherein the transgenic plant has an altered content of at least one aromatic amino acid compared to a corresponding non transgenic plant.

56. The transgenic plant of claim 55, wherein at least one of the CM activity and the PDT activity has reduced sensitivity to feedback inhibition by phenylalanine.

57. The transgenic plant of claim 55, wherein the polynucleotide encoding chorismate mutase and the polynucleotide encoding prephenate dehydratase are of bacterial origin.

58. The transgenic plant of claim 57, wherein the chorismate mutase and the prephenate dehydratase are encoded by a single polynucleotide encoding amino acids 1-300 of P-protein of E. coli (SEQ ID NO:1), the polynucleotide comprising a nucleic acids sequence as set forth in SEQ ID NO:10.

59. The transgenic plant of claim 58, wherein the polynucleotide further comprises a nucleic acid sequence encoding a plastid transit peptide, said polynucleotide having a nucleic acid sequence as set forth in SEQ ID NO:3.

60. The transgenic plant of claim 59, wherein the polynucleotide further comprises a nucleic acid sequence encoding hemagglutinin (HA) epitope tag as a selectable marker, and the selection is performed utilizing anti-HA antibodies, said polynucleotide having a nucleic acid sequence as set forth in SEQ ID NO:5.

61. The transgenic plant of claim 60, wherein the polynucleotide encodes a polypeptide having the amino acid sequence as set forth in SEQ ID NO:6.

62. The transgenic plant of claim 55, wherein the aromatic amino acid is selected from the group consisting of phenylalanine, tryptophan and tyrosine.

63. The transgenic plant of claim 62, having an increased amount of at least one of phenylalanine and tyrosine or a reduced amount of tryptophan compared to the corresponding non transgenic plant.

64. The transgenic plant of claim 63, having an increased amount of at least one catabolic product of phenylalanine compared to the corresponding non transgenic plant selected from the group consisting of phenethyl isothiocyanate, 2-phenylethyl glucosinolate, benzyl glucosinolate, phenylacetonitrile, sinapyl alcohol, coniferon, caffeol glucose, acetovanillone, vanillic acid glucoside, coumarate hexose, ferulate hexose or combinations thereof.

65. The transgenic plant of claim 63, having an increased amount of at least one catabolic product of tyrosine compared to the corresponding non transgenic plant selected from the group consisting of homogentistic acid, gamma-tocopherol, gamma-tocotrienol and combinations thereof.

66. The transgenic plant of claim 55, having a decreased amount of at least one catabolic product of phenylalanine compared to the corresponding non transgenic plant, wherein the catabolic product is phenylalanine 3-carboxy-2-hydroxy.

67. The transgenic plant of claim 63, having a decreased amount of at least one catabolic product of tryptophan compared to the corresponding non transgenic plant selected from the group consisting of 1-methoxy-3-indolylmethyl, 6-hydroxyindole-3-carboxylic acid 6-O-beta-D-glucopyranoside, 6-hydroxyindole-3-carboxylic acid beta-D-glucopyranosyl ester, 4-O-(indole-3-acetyl)-D-glucopyranose, tryptophan N-formyl-methyl ester and combinations thereof.

68. A plant seed produced by the transgenic plant of claim 55.

69. The plant seed of claim 68, wherein the seed is used for breeding a transgenic plant having an altered content of at least one aromatic amino acid selected from the group consisting of phenylalanine, tryptophan and tyrosine compared to a non transgenic plant.

70. A tissue culture comprising at least one transgenic cell of the plant of claim 55 or a protoplast derived therefrom.

71. The tissue culture of claim 70, wherein said tissue culture regenerates plants having an altered content of at least one aromatic amino acid selected from the group consisting of phenylalanine, tryptophan and tyrosine compared to a corresponding non transgenic plant.

72. A plant regenerated from the tissue culture of claim 70.

73. A method for altering the synthesis of at least one aromatic amino acid in a plant, comprising (a) transforming a plant cell with an exogenous polynucleotide encoding chorismate mutase and an exogenous polynucleotide encoding prephenate dehydratase; and (b) regenerating the transformed cell into a transgenic plant comprising at least one cell having an altered content of at least one aromatic acid compared to a corresponding cell of non transgenic plant.

74. The method of claim 73, wherein the aromatic amino acid is selected from the group consisting of phenylalanine, tryptophan and tyrosine.

75. The method of claim 74, wherein the transgenic plant has an increased amount of at least one of phenylalanine and tyrosine or a reduced amount of tryptophan compared to the non transgenic plant.

76. The method of claim 75, wherein the transgenic plant has an increased amount of at least one catabolic product of phenylalanine compared to the corresponding non transgenic plant selected from the group consisting of phenethyl isothiocyanate, 2-phenylethyl glucosinolate, benzyl glucosinolate, phenylacetonitrile, sinapyl alcohol, coniferon, caffeol glucose, acetovanillone, vanillic acid glucoside, coumarate hexose, ferulate hexose or combinations thereof.

77. The method of claim 75, wherein the transgenic plant has an increased amount of at least one catabolic product of tyrosine compared to the non transgenic plant, selected from the group consisting of homogentistic acid, gamma-tocopherol, gamma-tocotrienol and combinations thereof.

78. The method of claim 73, wherein the transgenic plant has a decreased amount of the catabolic product of phenylalanine compared to the corresponding non transgenic plant, wherein the catabolic product is phenylalanine 3-carboxy-2-hydroxy.

79. The method of claim 75, wherein the transgenic plant has a decreased amount of at least one catabolic product of tryptophan compared to the corresponding non transgenic plant, selected from the group consisting of 1-methoxy-3-indolylmethyl, 6-hydroxyindole-3-carboxylic acid 6-O-beta-D-glucopyranoside, 6-hydroxyindole-3-carboxylic acid beta-D-glucopyranosyl ester, 4-O-(indole-3-acetyl)-D-glucopyranose, tryptophan N-formyl-methyl ester and combinations thereof.

80. The method of claim 73, wherein at least one of the chorismate mutase activity and the prephenate dehydratase activity has reduced sensitivity to feedback inhibition by phenylalanine.

81. The method of claim 80, wherein the polynucleotide encoding chorismate mutase and the polynucleotide encoding prephenate dehydratase are of bacterial origin.

82. The method of claim 81, wherein the chorismate mutase and the prephenate dehydratase are encoded by a single polynucleotide encoding amino acids 1-300 of P-protein of E. coli (SEQ ID NO:1), the polynucleotide comprising a nucleic acids sequence as set forth in SEQ ID NO:10.

83. The method of claim 82, wherein the polynucleotide further comprises a nucleic acid sequence encoding a plastid transit peptide, and a nucleic acid sequence encoding the protein selectable marker hemagglutinin (HA) epitope tag, said polynucleotide having a nucleic acid sequence as set forth in SEQ ID NO:5.

84. The method of claim 83, wherein the polynucleotide encodes a polypeptide having the amino acid sequence as set forth in SEQ ID NO:6.

85. The method of claim 73, further comprising generating a seed from said transgenic plant.

86. The method of claim 85, wherein the seed is used for breeding a transgenic plant having an altered content of at least one aromatic amino acid selected from the group consisting of phenylalanine, tryptophan and tyrosine compared to a non transgenic plant.


SEQUENCE LISTING (15 sequences)

SEQ ID NO: 1 - protein, 386 aa, Escherichia coli
MTSENPLLALREKISALDEKLLALLAERRELAVEVGKAKLLSHRPVRDIDRERDLLERLITLGKAHHLDAHYITRLFQLIIEDSVLTQQALLQQHLNKINPHSARIAFLGPKGSYSHLAARQYAARHFEQFIESGCAKFADIFNQVETGQADYAVVPIENTSSGAINDVYDLLQHTSLSIVGEMTLTIDHCLLVSGTTDLSAINTVYSHPQPFQQCSKFLNRYPHWKIEYTESTSAAMEKVAQAKSPHVAALGSEAGGTLYGLQVLERIEANQRQNFTRFVVLARKAINVSDQVPAKTTLLMATGQQAGALVEALLVLRNHSLIMTRLESRPIHGNPWEEMFYLDIQANLESAEMQKALKELGEITRSMKVLGCYPSENVVPVDPT

SEQ ID NO: 2 - DNA, 1161 bp, Escherichia coli
atgacatcggaaaacccgttactggcgctgcgagagaaaatcagcgcgctggatgaaaaattattagcattactggcagagcgacgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccagcaacatctcaataaaattaatccgcactcagcacgcatcgcttttctcggccccaaaggttcttattcccatcttgcggcgcgccagtatgctgctcgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagtttgtcgattgttggcgagatgacgttaactatcgatcattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttgttaatggcgaccggacaacaagctggtgcactggttgaagcgttgctggtactgcgcaatcacagtctgattatgacccgtctggaatcacgtccgattcacggtaatccgtgggaagagatgttttatctggatattcaggccaatcttgaatcagcggaaatgcaaaaagcattgaaagagttaggggaaatcacccgttcgatgaaggtattgggctgttacccaagtgagaacgtagtgcctgttgatccaacctga

SEQ ID NO: 3 - DNA, 1092 bp, Artificial Sequence
  Synthetic polynucleotide
gtcgactagaaaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgccatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttgtaagaattc

SEQ ID NO: 4 - protein, 357 aa, Artificial Sequence
  Synthetic polypeptide
MASMISSSAVTTVSRASTVQSAAVAPFGGLKSMTGFPVKKVNTDITSITSNGGRVKCMPSENPLLALREKISALDEKLLALLAERRELAVEVGKAKLLSHRPVRDIDRERDLLERLITLGKAHHLDAHYITRLFQLIIEDSVLTQQALLQQHLNKINPHSARIAFLGPKGSYSHLAARQYAARHFEQFIESGCAKFADIFNQVETGQADYAVVPIENTSSGAINDVYDLLQHTSLSIVGEMTLTIDHCLLVSGTTDLSAINTVYSHPQPFQQCSKFLNRYPHWKIEYTESTSAAMEKVAQAKSPHVAALGSEAGGTLYGLQVLERIEANQRQNFTRFVVLARKAINVSDQVPAKTTL

SEQ ID NO: 5 - DNA, 2402 bp, Artificial Sequence
  Synthetic polynucleotide
ggtacccggggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactagaaaatggcttctatgatatcctcttcagctgtgactacagtcagccgtgcttctacggtgcaatcggccgcggtggctccattcggcggcctcaaatccatgactggattcccagttaagaaggtcaacactgacattacttccattacaagcaatggtggaagagtaaagtgcatgccatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttggaattcatcttttacccatacgatgttcctgactatgcgggctatccctatgacgtcccggactatgcaggatcctatccatatgacgttccagattacgctgctcagtagtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagt

SEQ ID NO: 6 - protein, 393 aa, Artificial Sequence
  Synthetic polypeptide
MASMISSSAVTTVSRASTVQSAAVAPFGGLKSMTGFPVKKVNTDITSITSNGGRVKCMPSENPLLALREKISALDEKLLALLAERRELAVEVGKAKLLSHRPVRDIDRERDLLERLITLGKAHHLDAHYITRLFQLIIEDSVLTQQALLQQHLNKINPHSARIAFLGPKGSYSHLAARQYAARHFEQFIESGCAKFADIFNQVETGQADYAVVPIENTSSGAINDVYDLLQHTSLSIVGEMTLTIDHCLLVSGTTDLSAINTVYSHPQPFQQCSKFLNRYPHWKIEYTESTSAAMEKVAQAKSPHVAALGSEAGGTLYGLQVLERIEANQRQNFTRFVVLARKAINVSDQVPAKTTLEFIFYPYDVPDYAGYPYDVPDYAGSYPYDVPDYAAQ

SEQ ID NO: 7 - DNA, 467 bp, Artificial Sequence
  Synthetic polynucleotide
ggtacccggggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgac

SEQ ID NO: 8 - DNA, 747 bp, Artificial Sequence
  Synthetic polynucleotide
tctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagt

SEQ ID NO: 9 - DNA, 28 bp, Artificial Sequence
  Synthetic polynucleotide
gtcgactagaaaatggcttctatgatat

SEQ ID NO: 10 - DNA, 900 bp, Artificial Sequence
  Synthetic polynucleotide
atgacatcggaaaacccgttactggcgctgcgagagaaaatcagcgcgctggatgaaaaattattagcattactggcagagcgacgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccagcaacatctcaataaaattaatccgcactcagcacgcatcgcttttctcggccccaaaggttcttattcccatcttgcggcgcgccagtatgctgctcgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagtttgtcgattgttggcgagatgacgttaactatcgatcattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttg

SEQ ID NO: 11 - DNA, 117 bp, Artificial Sequence
  Synthetic polynucleotide
gaattcatcttttacccatacgatgttcctgactatgcgggctatccctatgacgtcccggactatgcaggatcctatccatatgacgttccagattacgctgctcagtagtctaga

SEQ ID NO: 12 - DNA, 908 bp, Artificial Sequence
  Synthetic polynucleotide
gcatgccatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttggaattc

SEQ ID NO: 13 - DNA, 918 bp, Artificial Sequence
  Synthetic polynucleotide
aagcttatgggcatgccatcggaaaacccgttactggcgctgcgagagaaaatcagcgcgctggatgaaaaattattagcgttactggcagaacggcgcgaactggccgtcgaggtgggaaaagccaaactgctctcgcatcgcccggtacgtgatattgatcgtgaacgcgatttgctggaaagattaattacgctcggtaaagcgcaccatctggacgcccattacattactcgcctgttccagctcatcattgaagattccgtattaactcagcaggctttgctccaacaacatctcaataaaattaatccgcactcagcacgcatcgcttttctcggccccaaaggttcttattcccatcttgcggcgcgccagtatgctgcccgtcactttgagcaattcattgaaagtggctgcgccaaatttgccgatatttttaatcaggtggaaaccggccaggccgactatgccgtcgtaccgattgaaaataccagctccggtgccataaacgacgtttacgatctgctgcaacataccagcttgtcgattgttggcgagatgacgttaactatcgaccattgtttgttggtctccggcactactgatttatccgccatcaatacggtctacagccatccgcagccattccagcaatgcagcaaattccttaatcgttatccgcactggaagattgaatataccgaaagtacgtctgcggcaatggaaaaggttgcacaggcaaaatcaccgcatgttgctgcgttgggaagcgaagctggcggcactttgtacggtttgcaggtactggagcgtattgaagcgaatcagcgacaaaacttcacccgatttgtggtgttggcacgtaaagccattaacgtttctgaccaggttccggcgaaaacgacgttggaattc

SEQ ID NO: 14 - DNA, 41 bp, Artificial Sequence
  Synthetic primer
gccaagcttatgggcatgccatcggaaaacccgttactggc

SEQ ID NO: 15 - DNA, 35 bp, Artificial Sequence
  Synthetic primer
ccccggaattccaacgtcgttttcgccggaacctg